(19) United States
(12) Patent Application Publication
(10) Pub. No.: US 2005/0214818 A1
(43) Pub. Date: Sep. 29, 2005

(54) Manipulation of RNA interference through modulation of armitage activity

(75) Inventors: William E. Theurkauf, Sterling, MA (US); Phillip D. Zamore, Northboro, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/018,067

(22) Filed: Dec. 20, 2004

Related U.S. Application Data
(60) Provisional application No. 60/531,553, filed on Dec. 19, 2003.
(60) Provisional application No. 60/553,828, filed on Mar. 16, 2004.

(51) Int. Cl. C12Q 1/68

(52) U.S. Cl. 435/6

(57) ABSTRACT

The present invention relates to the discovery of both developmental and RNA interference roles for the armitage (armi) gene and its encoded polypeptide, Armitage (Armi). RNA interference requires a set of conserved cellular factors to suppress gene expression. These factors are the components of the RNAi pathway. The methods described herein are useful for modulating the RNAi pathway—both experimentally and therapeutically—by directedly impacting Armi activity.

### RELATED APPLICATIONS

This application claims the benefit of U.S. Ser. No. 60/553,828, entitiled “Manipulation of RNA Interference by Modulation of Armitage Activity,” filed on Mar. 16, 2004, and U.S. Ser. No. 60/531,553, entitiled “Manipulation of RNA Interference by Modulation of Armitage Activity,” filed on Dec. 19, 2003. The entire contents of these applications are hereby incorporated herein by reference.

### STATEMENT AS TO FEDERALLY SPONSORED RESEARCH

Funding for the work described herein was at least in part provided by the federal government (N.I.H. grants GM62862-01 and GM65236-01), which has certain rights in the invention.

### BACKGROUND OF THE INVENTION

All eukaryotic organisms share similar mechanisms for information transfer from DNA to RNA to protein. RNA interference (RNAi) represents an efficient mechanism for inactivating this transfer process for a specific targeted gene. Targeting is mediated by the sequence of the RNA molecule introduced to the cell. Double-stranded (ds) RNA can induce sequence-specific inhibition of gene function (genetic interference) in several organisms including the nematode, C. elegans (Fire et al., 1998, Nature 391:806-811), plants (including Arabidopsis thaliana), trypanosomes, Drosophila melanogaster, and planaria (Waterhouse et al., 1998, Proc. Natl. Acad. Sci. USA 94:13959-13964; Ngo et al., 1998, Proc. Natl. Acad. Sci. USA 95:14687-14692; Kennerdell and Carthew, 1998, Cell 95:1017-1026; Misquitta and Patterson, 1999, Proc. Natl. Acad. Sci. USA 96: 1451-1456; Sanchez-Alvorado and Newmark, 1999, Proc. Natl. Acad. Sci. USA 96:5049-5054). The discovery that dsRNA can induce genetic interference in organisms from several distinct phyla indicates a conserved mechanism and perhaps a conserved physiological role for the interference process. Indeed, several components of the RNAi pathway have recently been identified, including a number of gene products for which orthologs exist across a broad range of species (Hutvágner and Zamore, 2002; Findley et al., 2003; Williams and Rubin, 2002; Caudy et al., 2003). Yet in spite of recent progress towards a comprehensive identification of proteins that contribute to RNAi, the only RNAi component for which an epistatic and mechanistic role has thus far been detailed is Dicer (Bernstein et al., 2001; Billy et al., 2001; Grishok et al., 2001; Hutvágner et al., 2001; Ketting et al., 2001; Knight and Bass, 2001; Paddison et al., 2002; Park et al., 2002; Provost et al., 2002; Reinhart et al., 2002; Zhang et al., 2002; Doi et al., 2003; Myers et al., 2003).

Identification and improved understanding of the functional role of RNAi pathway components is required to realize the full therapeutic potential of the RNAi pathway. Directed modulation of RNAi pathway activity by chemical, pharmaceutical or genetic means is a valuable approach to enhance both basic biological research as well as treatment of disease states in model organisms and humans.

### SUMMARY OF THE INVENTION

The present invention relates to the discovery of both developmental and RNA interference roles for the armitage (armi) gene and its encoded polypeptide, Armitage (Armi). RNA interference requires a set of conserved cellular factors to suppress gene expression. These factors are the components of the RNAi pathway. The methods described herein are useful for modulating the RNAi pathway and may be used in conjunction with other methods involving the use of genetic inhibition by dsRNA (e.g., see U.S. Ser. No. 09/215,257, filed Dec. 18, 1998, incorporated herein by reference in its entirety).

RNAi pathway components (e.g., Armi) provide activities necessary for interference. These activities may be absent or not sufficiently activated in many cell types, including those of organisms such as humans in which genetic interference may have potential therapeutic value. Components of the RNAi pathway in D. melanogaster can be provided through transgenesis or as direct RNA:protein complexes to activate or directly mediate genetic interference in heterologous cells that are deficient in RNAi.

The Armi protein is a RNA helicase herein discovered to act at a critical stage of the RNAi pathway. Helicase activity has been provisionally attributed to Armi and its direct orthologs, SDE3 in Arabidopsis thaliana and Mov10 in mammals (the group comprising Drosophila Armi, Arabidopsis thaliana SDE3 and mammalian Mov10 are henceforth implied when referring to Armi, unless specified as the Drosophila form). Until the present invention, however, the role of Armi had not been ascribed to the RNAi pathway. The present discovery that Armi functions at a stage of the RNAi pathway at which RNA-induced silencing complex (RISC) assembly and activation occurs is of use in identifying agents that are capable of modulating the RNAi pathway.

In practice, precision modulation of RNAi pathway activity is provided by the methods of Armi activity modulation described herein. Accordingly, the instant invention features methods of detecting compounds, compositions, genes, gene products, polypeptides or polynucleotides that modulate Armi activity. The invention further features methods for detecting genes, gene products, polypeptides or polynucleotides that are influenced by Armi activity.

The instant invention relates to the discovery that a gene product named “Armitage” functions in the critical transitional stage between formation of siRNP complexes and production of an activated RISC. During this stage of the RNAi pathway, siRNAs are unwound and processed to select a single-stranded guide strand RNA; and loading of RISC with single-stranded guide siRNA, as well as RISC activation, occurs. Armi activity is thus required for proper functioning of RISC. The instant discovery that Armi functions specifically during this transitional phase of the RNAi pathway is of great value, as modulation of the activity of the Armi protein (Armi activity) enables relatively subtle alterations to be made to the overall functioning of the RNAi pathway, without directly impacting the specificity of the RNAi response. In one embodiment of the invention, therefore, methods of discovering RNAi modulatory compounds through observance of their effects on the Armi polypeptide are presented.

Another aspect of the invention is based, at least in part, on the discovery that the Armi polypeptide/Armi activity is required for proper localization of several developmental proteins, including the embryonic axis specification factors Vasa, Oskar, and Gurken. Accordingly, the instant invention provides methods for identifying embryonic developmental factors.

An additional aspect of the invention is based, at least in part, on the discovery that RNAi pathway activity may be monitored in ovary lysates via the methods described herein.

Other features and advantages of the invention will be apparent from the following detailed description and claims.

### BRIEF DESCRIPTION OF THE DRAWINGS

FIG. 1 shows that armi mutants disrupt axial patterning during early and late oogenesis. (A) Fluorescent in situ hybridization (FISH) localization of bicoid (bcd), oskar (osk) and gurken (grk) mRNAs in wild type and armi¹/armi¹ stage 9-10 egg chambers revealed mislocalization of osk and grk in armi mutants. fc, follicle cells; nc, nurse cell; O, oocyte. (A, inset) grk mRNA localized by colorimetric detection. (B) FISH localization of osk mRNA and Grk protein during early oogenesis, in wild type and armi mutant (armi^{72.1}/armi^{72.1}) stage 4 early oocytes, showed that osk mRNA and Grk protein failed to localize properly to the posterior of the oocyte. Egg chambers are oriented with anterior to the left and posterior to the right. The posterior cortex (arrowheads) and oocyte nucleus are indicated (n). Bars are 25 μm in (A) and 20 μm in (B).

FIG. 2 shows the armi genomic locus, the sequence of which revealed Armi to be a member of the SDE3/Mov10 family of RNA helicases. (A) A schematic representation of the armi locus is depicted, with thin lines representing introns; black boxes protein coding exons; black stippled boxes denoting regions containing 5′ and 3′ UTR sequences. The armi¹ allele contains a P-element inserted in the 5′ UTR (open triangle). Regions of the armi locus encoded by GM10845 and pKS-armi3 cDNAs are highlighted. The small arrows labeled 5 and 3 represent primers used for 5′ and 3′ RACE, and rt1 and rt2 denote primers used to amplify armi-cycJ read-through transcripts. (B) Schematic representation of the predicted Armi protein and the most closely related homologues is shown. Solid black areas indicate the conserved C-terminal region, which contains ATPase and helicase motifs. Percent identity and similarity (parentheses) to Armi are indicated. Hatched areas indicate regions of sequence divergence. (C) Conservation of motifs characteristic of the Upflp RNA helicase superfamily I in Armi/SDE3/Mov10 proteins. Predicted protein sequences from Armi (SEQ ID NO: 1), SDE3 (SEQ ID NO: 2), mouse Mov10 (SEQ ID NO: 3) and a human Mov10 (SEQ ID NO: 4) were aligned using the ClustalW program. Consensus ATPase, unwinding, and RNA binding sequences for the Upflp helicase superfamily I are indicated, and were generated based on the motifs in (Koonin, 1992; Linder and Daugeron, 2000; Tanner and Linder, 2001; Weng et al., 1996). (bolded black) denote identical amino acids; (gray) denote similar residues; “U”, a bulky hydrophobic residue (I, L, V, M, F, Y, W); “-” any residue.

FIG. 3 depicts a model for miRNA function in embryonic axis specification. The miRNA miR-280 forms a duplex with conserved sites in the 3′UTR of D. melanogaster and D. pseudoobscura osk mRNA (A) and kinesin heavy chain mRNA (B) and transcripts encoding additional cytoskeletal proteins that function during oogenesis (Stark et al., 2003). The RNA silencing system functions with miR-280 and other miRNAs to coordinately regulate translation of osk mRNA and additional cytoskeletal targets during the initial stages of embryonic axis specification.

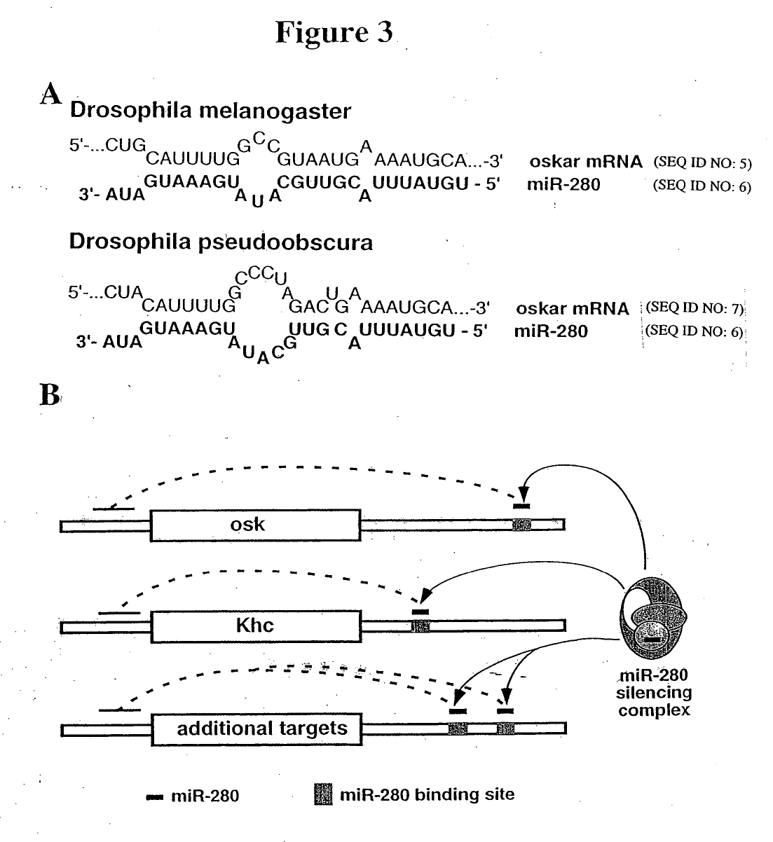

FIG. 4. Armi is required for Ste silencing in fly testes. The testes from wild type (A), armi¹ (B), armi^{72.1} (C) and armi^{rev39.2} flies (D) were stained for DNA (dull staining) and Ste protein (brighter staining), revealing mislocalization of Ste in armi mutants.

FIG. 5. Drosophila ovary lysate recapitulates RNAi in vitro. mRNA cleavage rate in ovary lysate using 5′ modified siRNA. Filled squares, 5′ PO₄ 2-(2′ dT); filled circles, 5′ PO₄ 2-(2′ riboU); open squares 5′ OH (2′ dT); open circles, 5′ OH (2′ riboU).

FIG. 6. armi ovary lysates are defective in RNAi. (A) Dorsal appendage phenotype was assessed for alleles of armi. (B) The fraction of target mRNA cleaved after 2 hours in an RNAi reaction using ovary lysates from armi alleles. (C) mRNA cleavage rate in wild-type and armi¹ ovary lysates programmed with siRNA.

FIG. 7. Armi and Aub are required for RISC assembly. (A) The RISC assembly assay of the present invention is depicted. (B) RISC assembly was tested with ovary lysates prepared from mutant alleles. C3 and C4 are RISC-like complexes formed only in the presence of siRNA. A 5′ phosphorylated, 2′ dT siRNA was used to maximize RISC assembly.

FIG. 8. Identification of intermediates in RISC assembly. (A) siRNA duplexes used for native gel analysis are shown. The strand that enters the RISC is the bottom strand for siRNAs 1, 3, 4 and 5, whereas the top strand enters the RISC for siRNA 2; deoxynucleotides (Thymine replacing Uracil) are present near the 5′ ends of the bottom strands of siRNAs 3, 4 and 5. ³²P-radiolabeled phosphates are indicated with an asterisk. (B) Diagram of the global kinetic model used for fitting of experimental RISC intermediate formation data. F, free siRNA. (C) Kinetic model of data for assembly of 5′-³²P-radiolabeled siRNA into protein complexes. At the 90 minute timepoint, RISC is most abundant, followed by complex A, free siRNA, and complex B, respectively. Arrow length corresponds to the relative amplitude of forward and reverse rate constants best describing the data.

FIG. 9. Dcr-2 binds tightly and crosslinks to siRNA. (A) The photocrosslinkable nucleoside 5-iodouridine, showing only the base for simplicity. (B) Schematic of the siRNA used for RNA-protein UV crosslinking, which revealed interaction with an ˜200 kDa protein (when the photocrosslinkable nucleoside was positioned at p20). (C) The ˜200 kDa protein crosslinked to siRNA copurifies with both Dicer activity and the peak of double-stranded siRNA (cf. FIG. 5C in Nykänen et al., 2001). (D) Schematic of an siRNA used for RNA-protein UV crosslinking, which failed to crosslink the ˜200 kDa protein. (E) Schematic of the 5′ photo-cleavable (PC) biotinylated siRNA duplex, which was used to identify Dcr-2 as the ˜200 kDa crosslinking protein.

FIG. 10. armi ovary lysates are defective in the ATP-dependent incorporation of single-stranded siRNA into RISC. (A) Results of target mRNA cleavage experiments for wild-type and mutant ovary lysates using single-stranded siRNA are shown. (B) Examination of the fraction of single-stranded siRNA remaining after incubation in embryo lysate in the presence (circles) or the absence (triangles) of ATP revealed that single-stranded siRNA was slightly more stable in the absence of ATP.

FIG. 11 presents a model for RNA silencing in Drosophila. Armi facilitates the ATP-dependent incorporation of siRNA into RISC, whereas Aubergine (Aub) is drawn as a RISC component.

### DETAILED DESCRIPTION

The instant invention primarily relates to the elucidation of a critical role for the Armitage protein in the RNA interference pathway. Armi was discovered to specifically function at the stage of the RNAi pathway in which RISC components and a double-stranded siRNA or miRNA generated by Dicer are transformed into an activated RISC, specifically a RISC that is loaded with a siRNA- or miRNA-derived single-stranded guide RNA that enables target RNA recognition by the activated RISC. Modulation of Armi activity can induce global alterations in the functionality of the RNAi pathway in a guide strand and target sequence-independent manner.

Accordingly, in one embodiment, the invention includes methods of identifying a compound or compounds capable of modulating Armi activity. Such a compound or compounds will, by way of exerting their effects on Armi, alter the overall functionality of the RNAi pathway in a guide strand and target sequence-independent manner.

In preferred embodiments, the Armi activity modulatory compound is identified following contact of the test compound with a cell or organism expressing an Armi polypeptide or fragment thereof; or following contact of the test compound with a composition or cell extract comprising an Armi polypeptide or fragment thereof.

A number of methods for detecting Armi activity may be employed in performing screening assays for an RNAi modulatory compound.

In one embodiment, the invention includes detection of Armi activity through detection of RISC assembly, RISC activation, or by means of detecting siRNA or miRNA processing for incorporation into RISC, or any combination these methods of detecting Armi activity.

In another embodiment, the invention involves detection of Armi activity by observing the interaction of Armi peptide with a peptide, peptides or fragments thereof of RISC or RISC-interacting factors, such as Argonaute (e.g. Aubergine), Stellate, Maelstrom and Dicer polypeptides or fragments thereof (and polypeptides and fragments of other RISC and RISC-related components).

In another embodiment, the invention includes detection of Armi activity through detection of RISC assembly, which is assessed by monitoring the interaction of an Armi polypeptide or fragment thereof with a peptide selected from the group of RISC and RISC-associated components, specifically Argonaute (e.g. Aubergine), Stellate, Maelstrom and Dicer polypeptides or fragments thereof (and polypeptides and fragments of other RISC and RISC-related components).

The invention also features detection of Armi activity, during performance of such screening assays for RNAi modulatory compounds, by means of detecting the interaction of an Armi peptide or fragment thereof with single-stranded products of siRNA unwinding.

Alterations in RISC activation may also be monitored as a means to detect changes in Armi activity induced by the test compound. Accordingly, in one embodiment, the invention includes detection of RISC activation as a means of assessing Armi activity.

In another embodiment, the invention includes monitoring the interaction of an Armi polypeptide or fragment thereof with single-stranded products of siRNA unwinding, in order to assess RISC activation, while performing screening assays for a test compound that modulates Armi activity.

In one embodiment of the instant invention, test compound(s) used in performing screening assays to identify those that modulate Armi activity, are selected from the group comprising a small molecule, peptide, polynucleotide, antibody or biologically active portion thereof, peptidomimetic, or non-peptide oligomer.

Armi activity may also be detected through use of a reporter RNA molecule. In one embodiment of the invention, RISC assembly or activation is detected by assessing the cleavage state of detectably labeled reporter RNA, while screening for a test compound that modulates Armi activity. In one embodiment of the instant invention, the reporter RNA is radioactively labeled. In another embodiment of the invention, the reporter RNA is fluorescently labeled.

In a specific embodiment, during screening for compounds that modulate Armi activity, the invention includes contact of the test compound(s) with an Armi-expressing cell from the following group: embryonic cells, ovarian cells Drosophila melanogaster cells, Drosophila melanogaster cell lines, mammalian cells, and mammalian cell lines.

In another specific embodiment, during screening for compounds that modulate Armi activity, the invention includes contact of the test compound(s) with an Armi-containing composition from the following group: embryonic cell lysates, ovarian cell lysates Drosophila melanogaster cell lysates, Drosophila melanogaster cell line lysates, mammalian cell lysates, and mammalian cell line lysates.

Screening for Armi modulatory compounds may also be performed in armi mutant or Armi-depleted cells or cell extracts. Accordingly, in one embodiment of the instant invention, armi mutant or Armi-depleted cells or cell extracts are contacted with a test compound and the impact of the test compound on Armi activity is examined.

In another embodiment, the invention includes contacting an armi mutant or knockout cell or cell extract expressing a mutant or wild-type form of Armi polypeptide or fragment thereof from a transgenic or exogenously-replicating vector with a test compound, and detecting the ability of the test compound to impact Armi activity, for the purpose of identifying an RNAi modulatory compound.

In an additional embodiment, the invention includes expressing a mutant or wild-type form of Armi polypeptide or fragment thereof from a transgenic or exogenously-replicating vector within an organism, embryo, cell or cell extract, and assessing the ability of an Armi polypeptide or fragment thereof to modulate RNA interference.

In another embodiment, the invention includes examination of the ability of an Armi polypeptide selected from the group consisting of Arabidopsis SDE3, Drosophila Armi, and mammalian Mov10, to modulate RNA interference, within the assay in which a mutant or wild-type form of Armi polypeptide or fragment thereof is expressed from a transgenic or exogenously-replicating vector within an organism, embryo, cell or cell extract.

In an additional embodiment, the organism, embryo, cell or cell extract expressing a mutant or wild-type form of Armi polypeptide or fragment thereof from a transgenic or exogenously-replicating vector, is an armi mutant or knockout organism, embryo, cell or cell extract.

In the instant invention, the discovery that armi mutant Drosophila embryos mislocalized and misexpressed a wide range of developmental factors illuminated a role for Armi activity in Drosophila embryonic development. Armi activity was also observed to be required for proper microtubule organization during embryonic development, consistent with an observed peripheral association between Armi and the Drosophila embryonic microtubule skeleton.

Accordingly, in one embodiment, the invention includes assessing at least one of the following for purposes of detecting modulation of Armi activity by test compound(s): disrupted localization of a polynucleotide or peptide, premature expression of a polynucleotide, and disrupted microtubule organization.

In another embodiment, the invention includes detection of the localization or abundance of specific developmental or RNAi factors as a means of detecting modulation of Armi activity by test compound(s). In this particular embodiment, the specific developmental or RNAi factors are selected from the group consisting of oskar mRNA, Oskar peptide, gurken mRNA, Gurken peptide, vasa mRNA, Vasa peptide, aubergine mRNA, Aubergine peptide, maelstrom mRNA, Maelstrom peptide, stellate mRNA and Stellate peptide.

The disrupted localization of embryonic developmental factors that the inventors observed in armi mutant embryos enables a method for identifying developmental factors as part of the instant invention. Specifically, in one embodiment, the invention includes comparison of armi mutant or Armi-depleted embryos, cells or cell extracts to wild-type or control embryos, cells or cell extracts, for the purpose of identifying developmental factors.

In one embodiment of such a developmental factor identification assay, potential developmental factors are specifically compared between armi mutant or Armi-depleted embryos, cells or cell extracts and wild-type or control embryos, cells or cell extracts.

In an additional embodiment of this developmental factor identification assay, the abundance of at least one potential developmental factor is compared between armi mutant or Armi-depleted embryos, cells or cell extracts and wild-type or control embryos, cells or cell extracts.

In another embodiment of the developmental factor identification assay, the localization or distribution of at least one potential developmental factor is compared between armi mutant or Armi-depleted embryos, cells or cell extracts and wild-type or control embryos, cells or cell extracts.

In a final embodiment of the developmental factor identification assay of the instant invention, the potential developmental factor that is examined constitutes an mRNA or mRNA fragment, or a polypeptide or fragment thereof.

One notable technological advance of the current invention is its elucidation of a method for performing RNAi pathway activity assays in dissected ovarian cells and cell lysates (past measurements of RNAi activity had been performed predominately in embryonic cells and cell lysates). In one embodiment, the invention therefore includes the detection of modulation of RNA interference in ovary lysates by the method of contacting a reaction mixture comprising ovary lysates with a test siRNA and evaluating the effect of the test siRNA on an indicator of RNA interference.

In one embodiment of this RNAi assay in ovarian lysates, the invention includes a detectably labeled reporter RNA, comprising a region complementary to a strand of the test siRNA, as an indicator of the functionality of the RNAi pathway.

The reporter RNA may be radioactively labeled in one embodiment of this RNAi ovarian lysate assay.

In another embodiment of the RNAi ovarian lysate assay, the reporter RNA may be fluorescently labeled.

In a specific embodiment of the RNAi ovarian lysate assay, assessment of the cleavage state of the detectably labeled reporter RNA is indicative of functionality of the RNAi pathway.

In another embodiment of the RNAi ovarian lysate assay, the reaction mixture that is contacted by a test siRNA specifically comprises Drosophila melanogaster ovary lysates.

In an additional embodiment of the RNAi ovarian lysate assay, the reaction mixture that is contacted by a test siRNA specifically comprises armi mutant or Armi-depleted Drosophila melanogaster ovary lysates.

In a final embodiment of the RNAi ovarian lysate assay, the reaction mixture comprises either a Drosophila melanogaster ovary lysate expressing Armi or a fragment thereof, or a Drosophila melanogaster armi mutant or Armi-depleted Drosophila melanogaster ovary lysate, with the composition further comprising a mammalian Mov10 polypeptide or fragment thereof.

Any compound(s) identified to alter Armi activity will be potentially attractive therapeutic entities, and in one embodiment, the invention therefore includes the method of administering compound(s) identified to alter RNA interference through impact on Armi activity, to a subject, at an effective concentration.

In another embodiment, a compound(s) that is identified to alter RISC activity through its impact on Armi activity is administered to a subject, at an effective concentration.

In an additional embodiment, the invention includes pharmaceutical compositions comprising any compound(s) identified as modulating RNA interference by the methods of this invention.

In another embodiment, the invention includes administration of a pharmaceutical composition comprising any compound(s) identified as modulating RNA interference by the methods of this invention, for the purpose of treating a disease caused by variant functioning of RNA interference.

In a final embodiment, the invention includes administration of a pharmaceutical composition comprising any compound(s) identified as modulating RNA interference by the methods of this invention, for the purpose of treating cancer.

So that the invention may be more readily understood, certain terms are first defined.

As used herein, both “protein” and “polypeptide” mean any chain of amino acids, regardless of length or post-translational modification (e.g., glycosylation or phosphorylation). Thus, the term “RNAi pathway polypeptide” includes a full-length, naturally occurring RNAi pathway polypeptide such as Armi protein, as well as recombinantly or synthetically produced polypeptides that correspond to a full-length, naturally occurring Armi protein, or to particular domains or portions of a naturally occurring RNAi pathway protein.

The language “an Armi polypeptide” as used herein includes molecules related to Armi, e.g., having certain shared structural and functional features. Armi polypeptides share amino acid sequence similarity with polypeptides identified in other species, with identified direct homologues of Armi including Arabidopsis thaliana SDE3 and mammalian Mov10 proteins. Armi polypeptides further have at least one Armi activity (e.g. helicase activity).

As used herein the term “compound” includes any reagent which is tested using the assays of the invention to determine whether it modulates an Armi polypeptide activity. More than one compound, e.g., a plurality of compounds, can be tested at the same time for their ability to modulate an Armi polypeptide activity in a screening assay.

In one embodiment, test compounds comprise any selection of the group consisting of a small molecule, a peptide, a polynucleotide, an antibody or biologically active portion thereof, a peptidomimetic, and a non-pepdide oligomer.

As used herein, “modulation” may constitute any alteration at any point in time of the relative activity or abundance of, for example, a gene, gene product, or pathway, as compared to wild-type levels. (Examples of such modulation include: gene knockouts, transgenic expression of a gene or mutant form of a gene, expression of a mutant form of a native gene, underexpression and overexpression of a gene.) An “RNAi modulatory compound” is therefore any compound capable of modulation in any manner of RNAi.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

The term “RNA interference” or “RNAi” (also referred to in the art as “gene silencing” and/or “target silencing”, e.g., “target mRNA silencing”), as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNAi agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNAi agent”, as used herein, refers to an RNA (or analog thereof), having sufficient sequence complementarity to a target RNA (i.e., the RNA being degraded) to direct RNAi. A RNAi agent having a “sequence sufficiently complementary to a target RNA sequence to direct RNAi” means that the RNAi agent has a sequence sufficient to trigger the destruction of the target RNA by the RNAi machinery (e.g., the RISC) or process. A RNAi agent having a “sequence sufficiently complementary to a target RNA sequence to direct RNAi” is also intended to mean that the RNAi agent has a sequence sufficient to trigger the translational inhibition of the target RNA by the RNAi machinery or process. A RNAi agent having a “sequence sufficiently complementary to a target RNA encoded by the target DNA sequence such that the target DNA sequence is chromatically silenced” means that the RNAi agent has a sequence sufficient to induce transcriptional gene silencing, e.g., to down-modulate gene expression at or near the target DNA sequence, e.g., by inducing chromatin structural changes at or near the target DNA sequence.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, an siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs).

As used herein, the term “microRNA” (“miRNA”) refers to an RNA (or RNA analog) comprising the product of an endogenous, non-coding gene whose precursor RNA transcripts can form small stem-loops from which mature miRNAs are cleaved by Dicer (Lagos-Quintana et al., 2001; Lau et al., 2001; Lee and Ambros, 2001; Lagos-Quintana et al., 2002; Mourelatos et al., 2002; Reinhart et al., 2002; Ambros et al., 2003; Brennecke et al., 2003; Lagos-Quintana et al., 2003; Lim et al., 2003a; Lim et al., 2003b). miRNAs are encoded in genes distinct from the mRNAs whose expression they control. Mature miRNAs represent the single stranded product of Dicer cleavage that then function as guide RNA fragments when incorporated into the RISC complex.

As used herein, the term “antisense strand” of an siRNA or RNAi agent refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” or “second strand” of an siRNA or RNAi agent refers to a strand that is complementary to the antisense strand or first strand. Antisense and sense strands can also be referred to as first or second strands, the first or second strand having complementarity to the target sequence and the respective second or first strand having complementarity to said first or second strand.

As used herein, the term “guide strand” refers to a strand of an RNAi agent, e.g., an antisense strand of an siRNA duplex, that enters into the RISC complex and directs cleavage of the target mRNA.

A “target gene” is a gene whose expression is to be selectively inhibited or “silenced.” This silencing is achieved by cleaving the mRNA of the target gene by an siRNA or miRNA, e.g., an siRNA or miRNA that is created from an engineered RNA precursor by a cell's RNAi system. One portion or segment of a duplex stem of the RNA precursor is an anti-sense strand that is complementary, e.g., sufficiently complementary to trigger the destruction of the desired target mRNA by the RNAi machinery or process, to a section of about 18 to about 40 or more nucleotides of the mRNA of the target gene.

As used herein, the term “RISC” refers to the proteins and single-stranded polynucleotides that interact to recognize target RNA molecules. Demonstrated components of RISC include the Argonaute proteins (e.g. Aubergine, Argonaute 2) and Dicer (e.g. Dcr-2). In the case of a RISC loaded with a single-stranded guide RNA derived from a siRNA, the RISC cleaves the target RNA molecule.

As used herein, the term “Armi activity” refers to any function carried out by the Armi polypeptide. In the instant invention, the Armi polypeptide is identified to function within the RNAi pathway, and specifically is necessary for RISC activity. In one embodiment, Armi activity comprises any of the following: RISC assembly, RISC activation, processing of siRNA or miRNA for incorporation into RISC. In another embodiment, Armi activity comprises interaction of Armi peptide with a peptide (or any combination of peptides) selected from the group comprising the molecular components of RISC (and RISC-related polypeptides and fragments thereof).

As used herein, a “test siRNA” is either a siRNA duplex or is a siRNA derived from an engineered precursor, and can be chemically synthesized or enzymatically synthesized. Such a test siRNA has the potential to direct RNA interference against a specific target gene.

The term “oligonucleotide” refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phophoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the “5′ end”, as in the 5′ end of an antisense strand, refers to the 5′ terminal nucleotides, e.g., between one and about 5 nucleotides at the 5′ terminus of the antisense strand. As used herein, the “3′ end”, as in the 3′ end of a sense strand, refers to the region, e.g., a region of between one and about 5 nucleotides, that is complementary to the nucleotides of the 5′ end of the complementary antisense strand.

As used herein, the term “isolated RNA” (e.g., “isolated shRNA”, “isolated siRNA” or “isolated RNAi agent”) refers to RNA molecules which are substantially free of other cellular material, or culture medium when produced by recombinant techniques, or substantially free of chemical precursors or other chemicals when chemically synthesized.

An embodiment of the present invention features mutant armi nucleic acid molecules or genes. The phrase “mutant nucleic acid molecule” or “mutant gene” as used herein, includes a nucleic acid molecule or gene having a nucleotide sequence which includes at least one alteration (e.g., substitution, insertion, deletion) such that the polypeptide or protein that may be encoded by said mutant exhibits an activity that differs from the polypeptide or protein encoded by the wild-type nucleic acid molecule or gene. Preferably, a mutant nucleic acid molecule or mutant gene (e.g., a mutant armi) encodes a polypeptide or protein having either reduced or enhanced Armi activity (e.g., displaying altered helicase or RISC activation activity) as compared to the polypeptide or protein encoded by the wild-type nucleic acid molecule or gene, for example, when assayed under similar conditions (e.g., assayed in cell lysates derived from cells cultured in parallel, differing only in Armitage genotype). A mutant gene also can encode no polypeptide or have a reduced or enhanced level of production of the wild-type polypeptide.

As used herein, a “reduced activity” is one that is at least 5% less than that of the polypeptide or protein encoded by the wild-type nucleic acid molecule or gene, preferably at least 5-10% less, more preferably at least 10-25% less and even more preferably at least 25-50%, 50-75% or 75-100% less than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, a “reduced activity” also includes an activity that has been deleted or “knocked out” (e.g., approximately 100% less activity than that of the polypeptide or protein encoded by the wild-type nucleic acid molecule or gene). Likewise, an “enhanced activity” is one that is at least 5% greater than that of the polypeptide or protein encoded by the wild-type nucleic acid molecule or gene, preferably at least 5-10% greater, more preferably at least 10-25% greater and even more preferably at least 25-50%, 50-75% or 75-100% greater or 100% or more greater (two-fold or greater elevated) than that of the polypeptide or protein encoded by the wild-type nucleic acid molecule or gene. Ranges intermediate to the above-recited values, e.g., 75-85%, 85-90%, 90-95%, are also intended to be encompassed by the present invention.

Activity can be determined according to any well accepted assay for measuring activity of a particular protein of interest. Activity can be measured or assayed directly, for example, measuring an activity of a protein isolated or purified from a cell. Alternatively, an activity can be measured or assayed within a cell or in an extracellular medium or in a crude extract of cells. Additionally, an Armi activity may also be measured in a whole organism.

It will be appreciated by the skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant nucleic acid or mutant gene (e.g., encoding a mutant polypeptide or protein), as defined herein, is readily distinguishable from a nucleic acid or gene encoding a protein homologue, as described above, in that a mutant nucleic acid or mutant gene encodes a protein or polypeptide having an altered activity, optionally observable as a different or distinct phenotype in a microorganism, cell or organism expressing said mutant gene or nucleic acid or producing said mutant protein or polypeptide (i.e., a mutant cell line) as compared to a corresponding microorganism, cell or organism expressing the wild-type gene or nucleic acid or producing said mutant protein or polypeptide. By contrast, a protein homologue has an identical or substantially similar activity, optionally phenotypically indiscernable when produced in a microorganism, cell or organism, as compared to a corresponding microorganism, cell or organism expressing the wild-type gene or nucleic acid. Accordingly it is not, for example, the degree of sequence identity between nucleic acid molecules, genes, protein or polypeptides that serves to distinguish between homologues and mutants, rather it is the activity of the encoded protein or polypeptide that distinguishes between homologues and mutants: homologues having, for example, low (e.g., 30-50% sequence identity) sequence identity yet having substantially equivalent functional activities, and mutants, for example sharing 99% sequence identity yet having dramatically different or altered functional activities. Exemplary homologues are set forth as SEQ ID NOs: SEQ ID NO: 1, SEQ ID NO: 2, SEQ ID NO: 3, SEQ ID NO: 4 (i.e., Armi polypeptides).

As used herein, an “armi mutant” cell, cell extract or organism, is any such entity harboring at least one mutant armi nucleic acid molecule or gene (including harboring a no form of the gene or polypeptide, e.g. an armi “knockout” state).

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected micleic acid sequence, all operably linked to the selected sequence, and may include an enhancer sequence.

As used herein, the term “autonomously replicating vector” refers to any vector capable of propagating or genomically integrating within target organism(s), that is also possible to propogate in at least one additional organism distinct from the target organism. Examples of autonomously replicating vectors include bacterial, yeast and mammalian shuttle vectors, YACs, BACs, cosmids, etc.

A gene “involved” in a disorder includes a gene, the normal or aberrant expression or function of which effects or causes a disease or disorder or at least one symptom of said disease or disorder.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Various aspects of the invention are described in further detail in the following subsections.

I. RISC and the RNAi Pathway

In eukaryotes, long double-stranded RNA (dsRNA) silences genes homologous in sequence, a process termed RNA interference (RNAi; Fire et al., 1998). RNAi and other examples of RNA silencing have been observed in animals, plants, protozoa, and fungi (Cogoni and Macino, 1997; Kennerdell and Carthew, 1998; Ngo et al., 1998; Waterhouse et al., 1998; Lohmann et al., 1999; Sánchez-Alvarado and Newmark, 1999; Wianny and Zernicka-Goetz, 2000; Caplen et al., 2001; Elbashir et al., 2001 a; Volpe et al., 2002; Schramke and Allshire, 2003). In plants, green algae, and invertebrates RNAi defends the genome against mobile genetic elements, such as transposons and viruses, whose expression and activity increase in RNAi-defective mutants (Ketting et al., 1999; Ratcliff et al., 1999; Tabara et al., 1999; Dalmay et al., 2000; Mourrain et al., 2000; Wu-Scharf et al., 2000; Aravin et al., 2001; Sijen and Plasterk, 2003). The RNAi pathway also regulates endogenous gene expression for at least one Drosophila gene, Stellate (Ste), which is targeted for destruction by dsRNA transcribed from the Suppressor-of-Stellate (Su(Ste)) locus (Aravin et al., 2001).

Long dsRNA is converted by Dicer, a multidomain ribonuclease III enzyme, into small interfering RNAs (siRNAs) (Zamore et al., 2000; Bernstein et al., 2001; Billy et al., 2001), which serve as the specificity determinants of the RNAi pathway (Hamilton and Baulcombe, 1999; Hammond et al., 2000; Zamore et al., 2000; Elbashir et al., 2001b). A related class of small RNAs, microRNAs (miRNAs), are generated by similar mechanisms from small stem-loop precursors transcribed from non-coding genes distinct from their regulatory targets (Grishok et al., 2001; Hutvágner et al., 2001; Ketting et al., 2001; Knight and Bass, 2001; Llave et al., 2002a; Park et al., 2002; Reinhart et al., 2002). In plants, miRNAs function like siRNAs, directing the endonucleolytic cleavage of target mRNAs, thereby regulating their spatial or temporal expression (Llave et al., 2002a; Llave et al., 2002b; Park et al., 2002; Reinhart et al., 2002; Rhoades et al., 2002; Palatnik et al., 2003; Tang et al., 2003; Xie et al., 2003). Both siRNAs and plant miRNAs direct mRNA cleavage as part of a protein-small RNA complex called the RNA-induced silencing complex (RISC; Hammond et al., 2000; Hammond et al., 2001). While animal miRNAs regulate endogenous gene expression through translational repression (Lee et al., 1993; Wightman et al., 1993; Olsen and Ambros, 1999; Brennecke et al., 2003), miRNA-mediated translational repression in animals also involves RISC activity (Hutvágner and Zamore, 2002; Mourelatos et al., 2002; Zeng et al., 2002; Doench et al., 2003; Saxena et al., 2003; Zeng et al., 2003). siRNAs and miRNAs act by guiding the RISC to the target RNA, and depending on the degree of complementarity between the siRNA/miRNA and their target, the RISC mediates either RNA degradation or translational repression (Doench et al., 2003; Hammond et al., 2000; Hutvágner and Zamore, 2002; Martinez et al., 2002; Zeng et al., 2002).

Members of the Argonaute family of proteins are core components of RISC or RISC-like complexes in flies (Hammond et al., 2001), worms (Tabara et al., 2002; Hutvágner et al., 2004), and humans (Caudy et al., 2002; Hutvágner and Zamore, 2002; Martinez et al., 2002; Mourelatos et al., 2002) and are required genetically for RNA silencing in every organism where their function has been studied (Tabara et al., 1999; Fagard et al., 2000; Grishok et al., 2000; Catalanotto et al., 2002; Caudy et al., 2002; Morel et al., 2002; Pal-Bhadra et al., 2002; Williams and Rubin, 2002; Doi et al., 2003).

A key step in RNA interference (RNAi) is the assembly of the RNA-induced silencing complex (RISC), a catalytically active protein-RNA complex that mediates target RNA cleavage. Each RISC contains one of the two strands of the small interfering RNA (siRNA) duplex that triggers RNAi. In vivo, initial generation of siRNAs is performed by the Dicer protein. siRNAs generated by Dicer are then incorporated into a complex termed the “siRNP”.

II. An Embryonic Developmental Role for Armitage

Discovery of an RNAi pathway role for Armi resulted from the present identification of armitage (armi) mutants via a screen for maternal effect genes in Drosophila. The screen revealed that loss of armitage exerted dramatic effects on the normally asymmetric distribution of a number of developmental factors. Normal asymmetric mRNA localization produces local protein concentrations that are critical to processes ranging from mating-type switching in yeast to synaptic plasticity in mammals (reviewed in Bashirullah et al., 1998; Bassell et al., 1999; Mohr and Richter, 2001). To produce protein at the right time and place within the cell, transcripts must be translationally silent during transport and remain silent until the protein products are needed. Embryonic axis specification in Drosophila is a well-studied developmental process that depends on spatial and temporal coordination of mRNA localization and translation (Cooperstock and Lipshitz, 2001; Riechmann and Ephrussi, 2001; van Eeden and St Johnston, 1999). bicoid (bcd) mRNA encodes the primary anterior morphogen and is localized to the anterior of the developing oocyte during stage 9 (Berleth et al., 1988; Driever and Nusslein-Volhard, 1988). However, bcd is not translated until egg activation, when the transcript is polyadenylated in the cytoplasm, recruited to polysomes, and translated to produce an anterior to posterior protein gradient (Driever and Nusslein-Volhard, 1988; Salles et al., 1994). Asymmetric localization of oskar (osk) mRNA during mid-oogenesis is essential for posterior patterning and for germ cell formation (Ephrussi et al., 1991; Kim-Ha et al., 1991; Lehmann and Nusslein-Volhard, 1986). osk transcript is produced throughout oogenesis, but remains translationally silent until localization to the oocyte posterior pole during stage 9 (Kim-Ha et al., 1995; Markussen et al., 1995; Rongo et al., 1995). Following localization and translational activation, Osk protein triggers the assembly of pole plasm, which specifies the germline and is required for abdominal patterning (Ephrussi et al., 1991; Ephrussi and Lehmann, 1992). Translational repression of osk during stages 7 and 8 is mediated by cis-elements in the osk 3′ UTR that are bound by Bruno (Kim-Ha et al., 1995; Webster et al., 1997), which interacts with Cup, an eIF4E binding protein (Nakamura et al., 2004). Cup is also required for osk translational silencing, and may function with Bruno to silence osk mRNA translation by blocking eIF4E interactions with other components of the translation initiation machinery (Nakamura et al., 2004; Wilhelm et al., 2003).

Unlike bcd and osk mRNAs, gurken (grk) mRNA is translated throughout oogenesis producing a TGFα-related growth factor that initiates two spatially and temporally distinct signaling events that specify the anterior-posterior (A/P) and dorsal-ventral (D/V) axes (Gonzalez-Reyes et al., 1995; Neuman-Silberberg and Schupbach, 1993; Neuman-Silberberg and Schupbach, 1996; Roth et al., 1995). During early oogenesis, microtubules originate from the posterior of the oocyte and mediate posterior localization of grk mRNA and Grk protein. Grk produced by the oocyte signals to the overlying follicle cells to induce posterior differentiation (Gonzalez-Reyes et al., 1995; Roth et al., 1995). During mid-oogenesis, the posterior follicle cells signal back to the oocyte, inducing reorganization of the oocyte microtubule network, which is essential for the asymmetric localization of bcd, osk and grk mRNAs (Gonzalez-Reyes et al., 1995; Roth et al., 1995). Following reorganization of the microtubule network, grk mRNA localizes to the dorsal-anterior corner of the oocyte (Neuman-Silberberg and Schupbach, 1993), and local Grk signaling induces dorsal differentiation of the adjacent follicle cells (Gonzalez-Reyes et al., 1995; Roth et al., 1995). The correct organization of oocyte microtubules early in oogenesis thus initiates a series of signaling events that specify the axes of the oocyte.

armitage (armi) was herein discovered to be required for posterior polarization of the microtubule cytoskeleton and translational silencing of osk mRNA during early oogenesis and for osk mRNA localization and posterior and dorsal-ventral patterning during mid-oogenesis. armi was also required for homology-dependent target RNA cleavage (RNAi) and efficient RISC assembly in ovary extracts. Three additional genes (Aubergine, an Argonaute protein, Spindle-E, a putative DEAD box helicase and Maelstrom) implicated in RNAi (Aravin et al., 2001; Findley et al., 2003; Kennerdell et al., 2002; Schmidt et al., 1999; Stapleton et al., 2001) were also herein shown to be required for osk mRNA translational silencing and microtubule reorganization during early oogenesis, indicating that the RNAi system is required for axial polarization of the oocyte. Additionally, Armi protein was demonstrated to be concentrated in the oocyte with osk mRNA. This asymmetric localization may spatially restrict RNA silencing activity and increase the efficiency of target recognition, and thus help establish the functional asymmetries that initiate embryonic axis specification.

III. Armi Homologues

Inspection of the primary amino acid sequence of Armi revealed that the armi gene encodes a putative RNA helicase. Previous genetic studies also reveal the importance of helicase-domain proteins in the RNAi pathway. Putative DEA(H/D) box helicases are required for post-transcriptional gene silencing (PTGS) in the green alga Chlamydomonas reinhardtii (Wu-Scharf et al., 2000) and RNAi in C. elegans (Tabara et al., 2002; Tijsterman et al., 2002a). In Drosophila, mutations in spindle-E (spn-E), a gene encoding a putative DEAD-box helicase, abrogate endogenous RNAi-based repression of the Ste locus and trigger expression of retrotransposon mRNA in the germline (Aravin et al., 2001; Stapleton et al., 2001). In cultured Drosophila S2 cells, the putative helicase Dmp68 is a component of affinity purified RISC (Ishizuka et al., 2002). Similarly, a putative DEAD-box RNA helicase, Gemin3, is a component of human RISC (Hutvágner and Zamore, 2002). Dicer, too, contains a putative ATP-dependent RNA helicase domain (Bernstein et al., 2001). Except for Dicer, no specific biochemical function in RNAi has been ascribed to any of these helicase proteins.

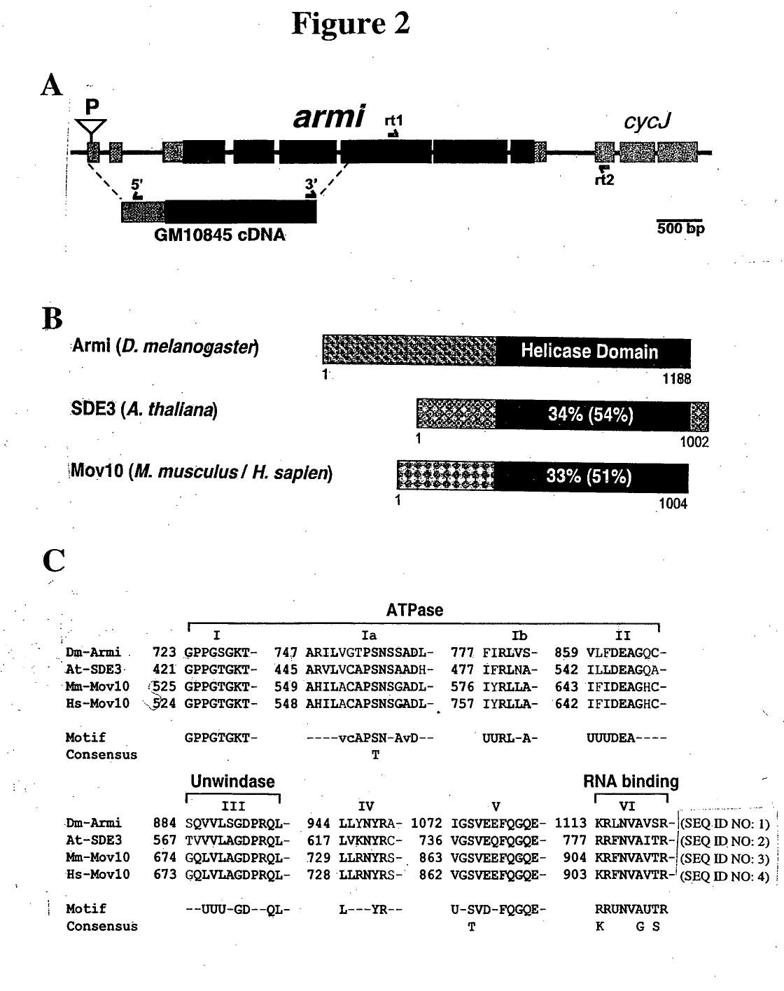

armitage (armi) was herein identified in a screen for maternal effect mutants that disrupt axis specification in Drosophila. Armitage protein (Armi) is a member of a family of putative ATP-dependent helicases distinct from the DEA(H/D) box proteins (Koonin, 1992). Armi is homologous across its putative helicase domain to SDE3, which is required for PTGS in Arabidopsis (Dalmay et al., 2001). Because PTGS in plants is mechanistically related to RNAi in animals, Armi was hypothesized to play a role in RNAi in flies. Armi is similarly homologous to mammalian Mov10 (Mooslehner et al., 1991) across its helicase domain (FIG. 2). This conserved helicase domain in Armi contains eight motifs characteristic of the Upflp family of ATP-dependent RNA helicases (FIG. 2; Koonin et al, 1992; Linder and Daugeron, 2000; Tanner and Linder, 2001; Weng et al., 1996). Herein, it was shown that armi is required for RNAi. armi mutant male germ cells failed to silence Stellate, a gene regulated endogenously by RNAi (Schmidt et al., 1999; Aravin et al., 2001; Stapleton et al., 2001), and lysates from armi mutant ovaries were defective for RNAi in vitro. Native gel analysis of protein-siRNA complexes in wild-type and armi mutant ovary lysates indicated that armi mutants supported early steps in the RNAi pathway, but were defective in the production of the RISC. These results indicated that armi (and armi homologues, e.g. SDE3 and Mov10) is required for the assembly of siRNA into functional RISC.

IV. RNA Molecules and Agents

The present invention features “small interfering RNA molecules” (“siRNA molecules” or “siRNA”), methods of making said siRNA molecules and methods (e.g., research and/or therapeutic methods) for using said siRNA molecules. An siRNA molecule of the invention is preferably a duplex consisting of a sense strand and complementary antisense strand, the antisense strand having sufficient complementarity to a target mRNA to mediate RNAi. Because only the antisense strand of an siRNA duplex incorporates into the RISC to mediate cleavage or silencing of the target mRNA, a single antisense strand capable of activating RISC (e.g., a stable form of the antisense strand) could mimic the functionality of the siRNA duplex. Preferably, the strands of an siRNA duplex are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 15-45 or 15-30 nucleotides. Even more preferably, the siRNA molecule has a length from about 16-25 or 18-23 nucleotides. The siRNA molecules of the invention further have a sequence that is “sufficiently complementary” to a target mRNA sequence to direct target-specific RNA interference (RNAi), as defined herein, i.e., the siRNA has a sequence sufficient to trigger the destruction of the target mRNA by the RNAi machinery or process.

siRNAs function as the specificity determinants of the RNAi pathway, where they act as guides to direct endonucleolytic cleavage of their target RNAs (Hamilton and Baulcombe, 1999; Hammond et al., 2000; Zamore et al., 2000; Elbashir et al., 2001b). The two strands of a siRNA are not equally eligible for assembly into RISC (Schwarz et al., 2003). Rather, both the absolute and relative stabilities of the base pairs at the 5′ ends of the two siRNA strands determine the degree to which each strand participates in the RNAi pathway. siRNA duplexes can be functionally asymmetric, with only one of the two strands able to trigger RNAi. Asymmetry is also the hallmark of a related class of small, single-stranded, non-coding RNAs, microRNAs (miRNAs).

In general, siRNA containing nucleotide sequences sufficiently identical to a portion of the target gene to effect RISC-mediated cleavage of the target gene are preferred. 100% sequence identity between the siRNA and the target gene is not required to practice the present invention. The invention can tolerate preferred sequence variations of the methods and compositions of the invention in order to enhance efficiency and specificity of RNAi. For example, siRNA sequences with insertions, deletions, and single point mutations relative to the target sequence can also be effective for inhibition. Alternatively, siRNA sequences with nucleotide analog substitutions or insertions can be effective for inhibition

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, between the siRNA antisense strand and the portion of the target gene is preferred. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41 (% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

The RNA molecules of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features small interfering RNAs (siRNAs) that include a sense strand and an antisense strand, wherein the antisense strand has a sequence sufficiently complementary to a target mRNA sequence to direct target-specific RNA interference (RNAi) and wherein the sense strand and/or antisense strand is modified by the substitution of internal nucleotides with modified nucleotides, such that in vivo stability is enhanced as compared to a corresponding unmodified siRNA. As defined herein, an “internal” nucleotide is one occurring at any position other than the 5′ end or 3′ end of nucleic acid molecule, polynucleotide or oligonucleoitde. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention the RNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O— and N-alkylated nucleotides, e.g., N6-methyl adenosine are suitable. It should be noted that the above modifications may be combined.

RNA may be produced enzymatically or by partial/total organic synthesis, any modified nibonucleotide can be introduced by in vitro enzymatic or organic synthesis. In one embodiment, an RNAi agent is prepared chemically. Methods of synthesizing RNA molecules are known in the art, in particular, the chemical synthesis methods as described in Verma and Eckstein (1998) Annul Rev. Biochem. 67:99-134. In another embodiment, an RNAi agent (e.g. a siRNA) is prepared enzymatically. For example, a ds-siRNA can be prepared by enzymatic processing of a long ds RNA having sufficient complementarity to the desired target mRNA. Processing of long ds RNA can be accomplished in vitro, for example, using appropriate cellular lysates and ds-siRNAs can be subsequently purified by gel electrophoresis or gel filtration. ds-siRNA can then be denatured according to art-recognized methodologies. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the siRNA can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands.

In one embodiment, the target mRNA of the invention specifies the amino acid sequence of a cellular protein (e.g., a nuclear, cytoplasmic, transmembrane, or membrane-associated protein). In another embodiment, the target mRNA of the invention specifies the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code. The following classes of proteins are listed for illustrative purposes: developmental proteins (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogene-encoded proteins (e.g., ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES); tumor suppressor proteins (e.g., APC, BRCA1, BRCA2, MADH4, MCC, NF I, NF2, RB I, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextriinases, DNA and RNA polymerases, galactosidases, glucanases, glucose oxidases, granule-bound starch synthases, GTPases, helicases, hernicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

In a preferred aspect of the invention, the target mRNA molecule of the invention specifies the amino acid sequence of a protein associated with a pathological condition. For example, the protein may be a pathogen-associated protein (e.g., a viral protein involved in immunosuppression of the host, replication of the pathogen, transmission of the pathogen, or maintenance of the infection), or a host protein which facilitates entry of the pathogen into the host, drug metabolism by the pathogen or host, replication or integration of the pathogen's genome, establishment or spread of infection in the host, or assembly of the next generation of pathogen. Alternatively, the protein may be a tumor-associated protein or an autoimmune disease-associated protein.

In one embodiment, the target mRNA molecule of the invention specifies the amino acid sequence of an endogenous protein (i.e., a protein present in the genome of a cell or organism). In another embodiment, the target mRNA molecule of the invention specified the amino acid sequence of a heterologous protein expressed in a recombinant cell or a genetically altered organism. In another embodiment, the target mRNA molecule of the invention specified the amino acid sequence of a protein encoded by a transgene (i.e., a gene construct inserted at an ectopic site in the genome of the cell). In yet another embodiment, the target mRNA molecule of the invention specifies the amino acid sequence of a protein encoded by a pathogen genome which is capable of infecting a cell or an organism from which the cell is derived.

By inhibiting the expression of such proteins, valuable information regarding the function of said proteins and therapeutic benefits which may be obtained from said inhibition may be obtained.

In one embodiment, siRNAs are synthesized either in vivo, in situ, or in vitro. Endogenous RNA polymerase of the cell may mediate transcription in vivo or in situ, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNAi agent (e.g. a siRNA). Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses an RNAi agent (e.g. a siRNA) from a recombinant construct may be produced by introducing the construct into a zygote, an embryonic stem cell, or another multipotent cell derived from the appropriate organism.

V. Short Hairpin RNAs (shRNAs)

In certain featured embodiments, the instant invention provides shRNAs having enhanced specificity or efficacy in mediating RNAi. In contrast to short siRNA duplexes, short hairpin RNAs (shRNAs) mimic the natural precursors of miRNAs and enter at the top of the RNAi pathway. For this reason, shRNAs are believed to mediate RNAi more efficiently by being fed through the entire natural RNAi pathway.

1. Short Hairpin RNAs that Generate siRNAs

shRNAs have a single strand that forms a duplex stem including two portions that are generally complementary, and a loop, that connects the two portions of the stem. In a preferred embodiment, short hairpin RNAs of the invention are artificial constructs engineered to deliver desired siRNAs.

In shRNAs of the instant invention, one portion of the duplex stem is a nucleic acid sequence that is complementary (or anti-sense) to the target mRNA. Thus, shRNAs include a duplex stem with two portions and a loop connecting the two stem portions. The two stem portions are about 18 or 19 to about 25, 30, 35, 37, 38, 39, or 40 or more nucleotides in length. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 micleotides. The overhanging nucleotides can include, for example, uracils (Us), e.g., all Us. The loop in the shRNAs can be 2, 3, 4, 5, 6, 7, 8, 9, or more, e.g., 15 or 20, or more nucleotides in length.

shRNAs of the invention include the sequences of the desired siRNA duplex. The desired siRNA duplex, and thus both of the two stem portions in the shRNA, are selected by methods known in the art.

A defining feature of the shRNAs of the invention is that as a consequence of their length, sequence, and/or structure, they do not induce sequence non-specific responses, such as induction of the interferon response or apoptosis, or that they induce a lower level of such sequence non-specific responses than long, double-stranded RNA (>150 bp) that has been used to induce RNAi. For example, the interferon response is triggered by dsRNA longer than 30 base pairs.

2. Transgenes Encoding Short Hairpin RNAs

The shRNAs of the invention can be synthesized by standard methods known in the art, e.g., by use of an automated DNA synthesizer (such as are commercially available from Biosearch, Applied Biosystems, etc.). The shRNAs can be used directly as described below or cloned into expression vectors by methods known in the field. The shRNAs can be delivered to cells in vitro or in vivo in which it is desired to target a specific mRNA for destruction. A number of methods have been developed for delivering DNA or RNA to cells. For example, for in vivo delivery, molecules can be injected directly into a tissue site or administered systemically. In vitro delivery includes methods known in the art such as electroporation and lipofection.

To achieve intracellular concentrations of the nucleic acid molecule sufficient to suppress expression of endogenous mRNAs, one can use, for example, a recombinant DNA construct in which the oligonucleotide is placed under the control of a strong Pol III (e.g., U6 or PolIII H1-RNA promoter) or Pol II promoter. The use of such a construct to transfect target cells in vitro or in vivo will result in the transcription of sufficient amounts of the shRNA to lead to the production of an siRNA that can target a corresponding mRNA sequence for cleavage by RNAi to decrease the expression of the gene encoding that mRNA. For example, a vector can be introduced in vivo such that it is taken up by a cell and directs the transcription of shRNA. Such a vector can remain episomal or become chromosomally integrated, as long as it can be transcribed to produce the desired shRNA.

Such vectors can be constructed by recombinant DNA technology methods known in the art. Vectors can be plasmid, viral, or other vectors known in the art such as those described herein, used for replication and expression in mammalian cells or other targeted cell types. The nucleic acid sequences encoding the shRNAs of the invention can be prepared using known techniques. For example, two synthetic DNA oligonucleotides can be synthesized to create a novel gene encoding the entire shRNA. The DNA oligonucleotides, which will pair, leaving appropriate ‘sticky ends’ for cloning, can be inserted into a restriction site in a plasmid that contains a promoter sequence (e.g., a Pol II or a Pol III promoter) and appropriate terminator sequences 3′ to the shRNA sequences (e.g., a cleavage and polyadenylation signal sequence from SV40 or a Pol III terminator sequence).

The invention also encompasses genetically engineered host cells that contain any of the foregoing expression vectors and thereby express the nucleic acid molecules of the invention in the host cell. The host cells can be cultured using known techniques and methods (see, e.g., Culture of Animal Cells (R. I. Freshney, Alan R. Liss, Inc. 1987); Molecular Cloning, Sambrook et al. (Cold Spring Harbor Laboratory Press, 1989)).

Successful introduction of the vectors of the invention into host cells can be monitored using various known methods. For example, transient transfection can be signaled with a reporter, such as a fluorescent marker, such as Green Fluorescent Protein (GFP). Stable transfection can be indicated using markers that provide the transfected cell with resistance to specific environmental factors (e.g., antibiotics and drugs), such as hygromycin B resistance, e.g., in insect cells and in mammalian cells.

3. Regulatory Sequences

The expression of the shRNAs of the invention is driven by regulatory sequences, and the vectors of the invention can include any regulatory sequences known in the art to act in mammalian cells, e.g., human or murine cells; in insect cells; in plant cells; or other cells. The term regulatory sequence includes promoters, enhancers, and other expression control elements. It will be appreciated that the appropriate regulatory sequence depends on such factors as the future use of the cell or transgenic animal into which a sequence encoding a shRNA is being introduced, and the level of expression of the desired shRNA. A person skilled in the art would be able to choose the appropriate regulatory sequence. For example, the transgenic animals described herein can be used to determine the role of a test polypeptide or shRNA in a particular cell type, e.g., a hematopoietic cell. In this case, a regulatory sequence that drives expression of the transgene ubiquitously, or a hematopoietic-specific regulatory sequence that expresses the transgene only in hematopoietic cells, can be used. Expression of the shRNA in a hematopoietic cell means that the cell is now susceptible to specific, targeted RNAi of a particular gene. Examples of various regulatory sequences are described below.

The regulatory sequences can be inducible or constitutive. Suitable constitutive regulatory sequences include the regulatory sequence of a housekeeping gene such as the α-actin regulatory sequence, or may be of viral origin such as regulatory sequences derived from mouse mammary tumor virus (MMTV) or cytomegalovirus (CMV).

Alternatively, the regulatory sequence can direct transgene expression in specific organs or cell types (see, e.g., Lasko et al., 1992, Proc. Natl. Acad. Sci. USA 89:6232). Several tissue-specific regulatory sequences are known in the art including the albumin regulatory sequence for liver (Pinkert et al., 1987, Genes Dev. 1:268276); the endothelin regulatory sequence for endothelial cells (Lee, 1990, J. Biol. Chem. 265:10446-50); the keratin regulatory sequence for epidermis; the myosin light chain-2 regulatory sequence for heart (Lee et al., 1992, J. Biol. Chem. 267:15875-85), and the insulin regulatory sequence for pancreas (Bucchini et al., 1986, Proc. Natl. Acad. Sci. USA 83:2511-2515), or the vav regulatory sequence for hematopoietic cells (Oligvy et al., 1999, Proc. Natl. Acad. Sci. USA 96:14943-14948). Another suitable regulatory sequence, which directs constitutive expression of transgenes in cells of hematopoietic origin, is the murine MHC class I regulatory sequence (Morello et al., 1986, EMBO J. 5:1877-1882). Since NMC expression is induced by cytokines, expression of a test gene operably linked to this regulatory sequence can be upregulated in the presence of cytokines.

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals such as mice, include regulation by ecdysone, by estrogen, progesterone, tetracycline, chemical inducers of dimerization, and isopropyl-beta-D1-thiogalactopyranoside (IPTG) (collectively referred to as “the regulatory molecule’). Each of these expression systems is well described in the literature and permits expression of the transgene throughout the animal in a manner controlled by the presence or absence of the regulatory molecule. For a review of inducible expression systems, see, e.g., Mills, 2001, Genes Devel. 15:1461-1467, and references cited therein.

The regulatory elements referred to above include, but are not limited to, the cytomegalovirus hCMV immediate early gene, the early or late promoters of SV40 adenovirus (Bernoist et al., Nature, 290:304, 1981), the tet system, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors. Additional promoters include the promoter contained in the 3′ long terminal repeat of Rous sarcoma virus (Yamamoto et al., Cell 22:787-797, 1988); the herpes thymidine kinase promoter (Wagner et al., Proc. Natl. Acad. Sci. USA 78:1441, 1981); or the regulatory sequences of the metallothionein gene (Brinster et al., Nature 296:39, 1988).

VI. Methods of Introducing RNAs, Vectors, and Host Cells

Physical methods of introducing nucleic acids include injection of a solution containing the nucleic acid (e.g., RNAi agent), bombardment by particles covered by the nucleic acid (e.g., RNAi agent), soaking the cell or organism in a solution of the nucleic acid (e.g., RNAi agent), or electroporation of cell membranes in the presence of the nucleic acid (e.g., RNAi agent). A viral construct packaged into a viral particle would accomplish both efficient introduction of an expression construct into the cell and transcription of a RNAi agent encoded by the expression construct. Other methods known in the art for introducing nucleic acids to cells may be used, such as lipid-mediated carrier transport, chemical-mediated transport, such as calcium phosphate, and the like. Thus the nucleic acid (e.g., RNAi agent) may be introduced along with components that perform one or more of the following activities: enhance nucleic acid (e.g., RNAi agent) uptake by the cell, inhibit annealing of single strands, stabilize the single strands, or other-wise increase inhibition of the target gene.

The nucleic acid (e.g., RNAi agent) may be directly introduced into the cell (i.e., intracellularly); or introduced extracellularly into a cavity, interstitial space, into the circulation of an organism, introduced orally, or may be introduced by bathing a cell or organism in a solution containing the nucleic acid (e.g., RNAi agent). Vascular or extravascular circulation, the blood or lymph system, and the cerebrospinal fluid are sites where the nucleic acid (e.g., RNAi agent) may be introduced.

The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals. Fungi include organisms in both the mold and yeast morphologies. Plants include Arabidopsis thaliana thaliana; field crops (e.g., alfalfa, barley, bean, corn, cotton, flax, pea, rape, nice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, black-berry, blueberry, cacao, cherry, coconut, cranberry, date, faJoa, filbert, grape, grapefr-uit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber). Examples of vertebrate animals include fish, mammal, cattle, goat, pig, sheep, rodent, hamster, mouse, rat, primate, and human; invertebrate animals include nematodes, other worms, drosophila, and other insects.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of RNAi agent delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of a RNAi agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNAi agent may be introduced in an amount which allows delivery of at least one copy per cell. Higher doses (e.g., at least 5, 10, 100, 500 or 1000 copies per cell) of material may yield more effective inhibition; lower doses may also be useful for specific applications.

VII. Methods of Treatment:

The present invention provides for both prophylactic and therapeutic methods of treating a subject at risk of (or susceptible to) a disorder or having a disorder associated with aberrant or unwanted target gene expression or activity. “Treatment”, or “treating” as used herein, is defined as the application or administration of a therapeutic agent (e.g., a RNAi modulatory agent, Armi modulatory agent, RNAi agent, or vector or transgene encoding same) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted target gene expression or activity, by administering to the subject a therapeutic agent (e.g., a RNAi modulatory agent, Armi modulatory agent, RNAi agent, or vector or transgene encoding same). Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

2. Therapeutic Methods

Another aspect of the invention pertains to methods of modulating target gene expression, protein expression or activity for therapeutic purposes. Accordingly, in an exemplary embodiment, the modulatory method of the invention involves contacting a cell capable of expressing target gene with a therapeutic agent (e.g., a RNAi modulatory agent, Armi modulatory agent, RNAi agent, or vector or transgene encoding same) that is specific for the target gene or protein such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

3. Pharmacogenomics

Therapeutic agents can be tested in an appropriate animal model. For example, a modulatory agent or RNAi agent (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

VIII. Pharmaceutical Compositions

The invention pertains to uses of the above-described agents for therapeutic treatments as described infra. Accordingly, the modulators or agents of the present invention can be incorporated into pharmaceutical compositions suitable for administration. Such compositions typically comprise the modulatory compound or RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

IX. Knockout and/or Knockdown Cells or Organisms

A further preferred use for the Armi activity modulatory molecules and compounds of the present invention (or RNAi agents or vectors or transgenes encoding same) is a functional analysis to be carried out in eukaryotic cells, or eukaryotic non-human organisms, preferably mammalian cells or organisms and most preferably human cells, e.g. cell lines such as HeLa or 293 or rodents, e.g. rats and mice. By administering a suitable RNAi agent (e.g. a siRNA) which is sufficiently complementary to a target mRNA sequence to direct target-specific RNA interference, a specific knockout or knockdown phenotype can be obtained in a target cell, e.g. in cell culture or in a target organism. Molecules or compounds of the instant invention that modulate Armi activity can either accentuate or counter such phenotypes.

Thus, a further subject matter of the invention is a eukaryotic cell or a eukaryotic non-human organism exhibiting a target gene-specific knockout or knockdown phenotype comprising a fully or at least partially deficient expression of at least one endogeneous target gene wherein said cell or organism is transfected with at least one vector comprising DNA encoding a RNAi agent (e.g. a siRNA) capable of inhibiting the expression of the target gene. It should be noted that the present invention allows a target-specific knockout or knockdown of several different endogeneous genes due to the specificity of the RNAi agent (e.g. a siRNA).

Gene-specific knockout or knockdown phenotypes of cells or non-human organisms, particularly of human cells or non-human mammals may be used in analytic procedures, e.g. in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g. an affinity tag, particularly a multiple affinity tag.

X. Mutant, Transgenic and Armi-Depleted Cells and Organisms

In one aspect of the instant invention, cells or organisms that are mutant for the Armitage gene are employed. Mutated forms of Armitage differ from the endogenous target gene in that they encode a gene product which differs from the endogenous gene product on the amino acid level by substitutions, insertions and/or deletions of single or multiple amino acids. Whereas variant forms usually have the same biological activity as the endogenous target gene, mutated target gene ussully have a biological activity which differs from the biological activity of the endogeneous target gene, e.g., a partially deleted activity, a completely deleted activity, an enhanced activity etc.

Cells and organisms harboring mutant forms of Armitage can be designed by standard methods for producing, e.g. knockout and transgenic cells and organisms, cells or organisms expressing mutant or wild type forms of Armitage from autonomously-replicating vectors.

Armitage, shRNAs and selected RNAi agents (e.g. a siRNA) of the invention can be expressed in transgenic animals. These animals represent a model system for the study of disorders that are caused by, or exacerbated by, overexpression or underexpression (as compared to wildtype or normal) of Armi, or of nucleic acids (and their encoded polypeptides) targeted for destruction by the RNAi agents, e.g., siRNAs and shRNAs, and for the development of therapeutic agents that modulate the expression or activity of Armi, or of nucleic acids or polypeptides targeted for destruction.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). Invertebrates such as Caenorhabditis elegans or Drosophila can be used as well as non-mammalian vertebrates such as fish (e.g., zebrafish) or birds (e.g., chickens).

A transgenic founder animal can be identified based upon the presence of a transgene (e.g. Armitage) in its genome, and/or expression of the transgene in tissues or cells of the animals, for example, using PCR, Northern analysis or immunoprecipitation.

In target gene-specific activation of the RNAi pathway, shRNAs with stems of 18 to 30 nucleotides in length are preferred for use in mammals, such as mice. A transgenic founder animal can be identified based upon the presence of a transgene that encodes the new RNA precursors in its genome, and/or expression of the transgene in tissues or cells of the animals, for example, using PCR or Northern analysis. Expression is confirmed by a decrease in the expression (RNA or protein) of the target sequence.

A transgenic founder animal can be used to breed additional animals carrying the transgene. Moreover, transgenic animals carrying a transgene encoding the RNA precursors can further be bred to other transgenic animals carrying other transgenes. In addition, cells obtained from the transgenic founder animal or its offspring can be cultured to establish primary, secondary, or immortal cell lines containing the transgene.

In addition to generation of knockout cells and organisms as a method of depleting Armi from the “Armi-depleted embryos, cells, or cell extracts” of the invention, Armi can be depleted, for example, by methods such as immunodepletion, and other art-recognized methods of lowering the relative abundance of a protein in an organism, cell, or cell extract.

One aspect of the instant invention relates to complementation of an armi mutation in a cell or organism by way of introduction of a mutant or wild type form of Armi (or a polypeptide thereof) or an Armi homolog (or polypeptide thereof). Such complementation procedures are well established in the art.

XI. Functional Genomics and/or Proteomics

One application for the cell or organism of the invention is the analysis of gene expression profiles and/or proteomes. An analysis of a variant or mutant form of one or several target proteins is carried out, wherein said variant or mutant forms are reintroduced into the cell or organism by an exogenous target nucleic acid as described above. The combination of knockout of an endogeneous gene and rescue by using mutated, e.g. partially deleted exogenous target has advantages compared to the use of a knockout cell. Further, this method is particularly suitable for identifying functional domains of the targeted protein. In a further preferred embodiment a comparison, e.g. of gene expression profiles and/or proteomes and/or phenotypic characteristics of at least two cells or organisms is carried out. These organisms are selected from: (i) a control cell or control organism without target gene inhibition, (ii) a cell or organism with target gene inhibition and (iii) a cell or organism with target gene inhibition plus target gene complementation by an exogenous target nucleic acid.

Furthermore, the RNA knockout complementation method may be used for is preparative purposes, e.g. for the affinity purification of proteins or protein complexes from eukaryotic cells, particularly mammalian cells and more particularly human cells. In this embodiment of the invention, the exogenous target nucleic acid preferably codes for a target protein which is fused to art affinity tag. This method is suitable for functional proteome analysis in mammalian cells, particularly human cells.

Another utility of the present invention could be a method of identifying gene function in an organism comprising the use of a RNAi agent (e.g. a siRNA) to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects. A simple assay would be to inhibit gene expression according to the partial sequence available from an expressed sequence tag (EST). Functional alterations in growth, development, metabolism, disease resistance, or other biological processes would be indicative of the normal role of the EST's gene product.

The ease with which RNA can be introduced into an intact cell/organism containing the target gene allows the present invention to be used in high throughput screening (HTS). Solutions containing RNAi agents (e.g. siRNAs) that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified RNA can be fed directly to, injected into, the cell/organism containing the target gene. Alternatively, the RNAi agent (e.g. a siRNA) can be produced from a vector, as described herein. Vectors can be injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a colorimetric, fluorogenic, or luminescent signal in response to a regulated promoter (e.g., transfected with a reporter gene construct) can be assayed in an HTS format.

The present invention may be useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of an RNAi agent (e.g. a siRNA) at specific times of development and locations in the organism without introducing permanent mutations into the target genome.

XII. Screening Assays

The preferred methods of the invention relate to identifying and/or characterizing potential pharmacological agents, e.g. identifying new pharmacological agents from a collection of test substances and/or characterizing mechanisms of action and/or side effects of known pharmacological agents.

The invention provides methods (also referred to herein as “screening assays”) for identifying modulators, i.e., candidate or test compounds or agents (e.g., peptides, peptidomimetics, peptoids, small molecules or other drugs) which (a) have a modulatory (e.g., stimulatory or inhibitory) effect on the activity of Armi or, more specifically, (b) have a modulatory effect on the interactions of Armi with one or more of its natural substrates (e.g., peptide, protein, hormone, co-factor, or nucleic acid), or (c) have a modulatory effect on the expression of Armi, or (d) elicit a modulatory effect on RNAi pathway activity by impacting Armi activity. Such assays typically comprise a reaction between a wild type or mutant form of Armi (or polypeptide thereof) or cell, organism, or cell extract expressing a wild type or mutant form of Armi (or polypeptide thereof) and one or more assay components. The other components may be either the test compound itself, or any combination of test compound, test siRNA, wild type or mutant test forms of Armitage, and binding partners of Armitage.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the library is a natural product library, e.g., a library produced by a bacterial, fungal, or yeast culture. In another preferred embodiment, the library is a synthetic compound library.

Genomic libraries may also be utilized in aspects of the invention related to identification of developmental factors affected by modulation of Armi activity. Generation of such genomic libraries is well-documented in the art.

In one embodiment, the invention provides assays for screening candidate or test compounds which bind to Armi or a biologically active portion thereof. Determining the ability of the test compound to directly bind to a protein can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the expression of Armi or Armi activity, and thereby elicit an effect on RNAi pathway activity. Screens for compounds that modulate Armi and/or RNAi activity can be performed in a number of ways. In one aspect of the invention, assessment of the effect of a potential Armi or RNAi modulatory agent, e.g. a test compound, on Armi or RNAi activity, may be examined by subjecting the compositions, cells, and cell lysates of the instant invention to the potential Armi or RNAi modulatory agent in a tube or related type of vessel. Screening of a library of compounds for the purpose of performing a high-throughput examination of the effect(s) of a large number of compounds on the Armi or RNAi activity of the instant invention's compositions, cells, organisms and lysates can also be performed, for example, in microwells. Armi-containing compositions, cells, organisms, and lysates of the invention can also be screened against a matrix of test compounds to identify compounds capable of modulating Armi activity.

Monitoring of Armi activity in such screening assays can involve a number of assay readouts, e.g., detection of helicase activity, RISC assembly, RISC activation, Armi processing of siRNA or miRNA, the ability of a test siRNA to effect cleavage of a target mRNA or detectably labeled reporter target RNA.

Detection of Armi interaction with other polypeptides and nucleic acids can be performed, for example, through use of chemical crosslinking and immunoprecipitation methods, isolation of complexes through affinity column methodologies, or other art-recognized methods.

Screens for developmental factors whose localization is disrupted in an armi mutant may involve, for example, immunofluorescent visualization of the distribution of potential developmental factors in a number of embryos.

In one embodiment of the invention, a screen for a mutant form of Armi capable of imparting modified Armi activity can be performed. Generation of mutant forms of armi may be performed, for example, through traditional mutagenic methods, e.g. EMS-mediated mutagenesis of organisms, or through techniques such as error-prone PCR and site-directed mutagenesis.

The skilled artisan will appreciate that the enumerated organisms are also useful for practicing other aspects of the invention, e.g., making transgenic organisms as described infra.

The invention is further described in the Examples below which describe methods of identifying genes and processes affected by Armi activity in the RNAi pathway and methods of identifying compounds that modulate Armi activity, thereby modulating RNAi pathway activity. The following examples should not be construed as limiting. The contents of all references, patents and published patent applications cited throughout this application are incorporated herein by reference.

### EXAMPLES

### Example I

### Genetic and Molecular Characterization of the Armitage Gene

To identify new genes involved in embryonic axis specification, a collection of P-element transposon induced maternal effect lethal mutations was screened for impaired localization of the pole plasm component Vasa and for disruption of dorsal-ventral (D/V) patterning as indicated by defects in the dorsal appendages on the eggshell. This screen identified a new mutation that produced variable appendage defects and significantly reduced Vasa accumulation at the posterior pole (not shown). Molecular and genetic analyses demonstrated that the mutation was not in a previously identified gene (see below). The locus was named armitage, after the navigator on Robert Falcon Scott's failed Discovery expedition to the South Pole.

Excision of the P-element in the original mutant, armi¹, produced new armi alleles and revertant chromosomes that presumably restore gene function through precise transposon excision. Of the forty lines generated, 21 (52%) fully complemented armi¹ and were homozygous fertile with normal embryonic patterning. The remaining lines failed to complement armi¹, indicating that imprecise P-element excision had generated new armi mutations. Most of these mutations were homozygous viable and female sterile, and the mutant females produced eggs with intermediate to weak defects in D/V patterning, indicating they were partial loss-of-function alleles. However, armi^{72.1} appeared to be a strong loss-of-function allele. Homozygous armi¹ females produced eggs where 67% (n=608) showed strong D/V patterning defects as indicated by a complete lack of dorsal appendages. By contrast, 92% (n=885) of eggs deposited by armi^{72.1} females completely lacked dorsal appendages, and 35% of these were also collapsed. armi^{72.1} over the deficiency Df(3L)E1 showed a similar range of defects, indicating that armi^{72.1} is a strong loss-of-function mutation. However, both armi¹ and armi^{72.1} ovaries produce low levels of transcript, indicating that neither allele is functionally null (see below).

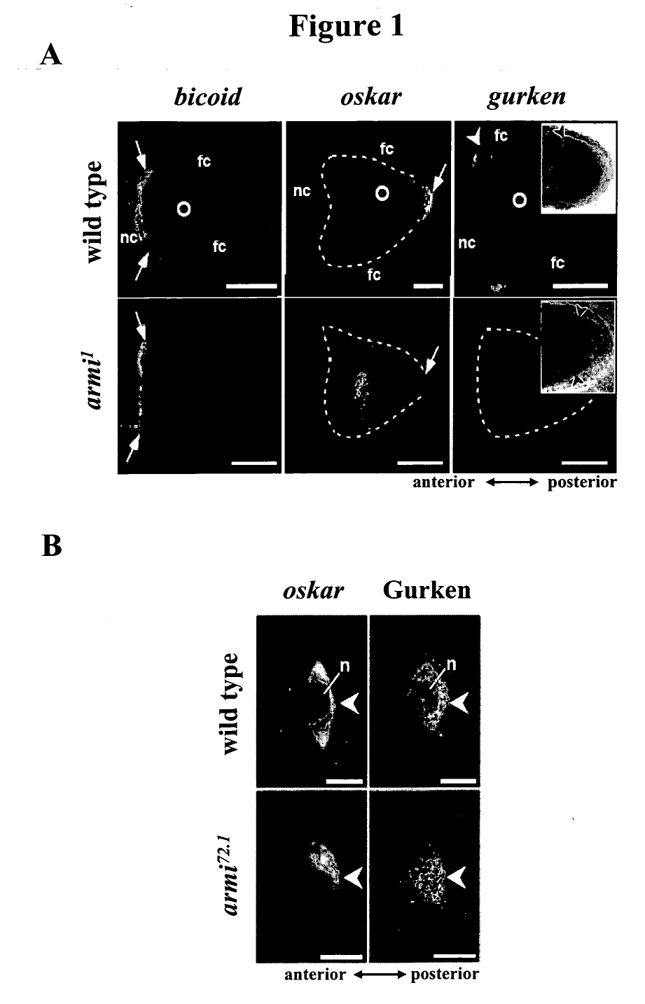

To further characterize armi patterning defects, fluorescent in situ hybridization (FISH) was performed for the three asymmetrically localized mRNAs that specify the anterior, posterior and dorsal regions of the oocyte. In wild type stage 9 to 11 oocytes, bcd mRNA accumulated along the anterior cortex (St Johnston et al., 1989) and osk mRNA localized to the posterior pole (FIG. 1A) (Castagnetti and Ephrussi, 2003; Kim-Ha et al., 1993). In armi mutants, bcd mRNA showed a wild type anterior distribution (FIG. 1A). However, osk RNA was either dispersed throughout the ooplasm or concentrated within the oocyte interior in 85% (n=52) of armi¹ egg chambers (FIG. 1A) and 90% (n=69) of armi^{72.1} egg chambers. The remaining egg chambers displayed weak posterior localization. osk mRNA consistently showed normal posterior localization in armi^{rev39.2} revertants. During stages 2 through 6, osk mRNA accumulates at the posterior of wild type oocytes (FIG. 1B) (Castagnetti and Ephrussi, 2003; Kim-Ha et al., 1993). In armi mutants, osk mRNA was transported to the oocyte during these early stages, but it did not accumulate at the posterior cortex (FIG. 1B).

During stages 2 to 7, grk mRNA and protein also accumulate at the oocyte posterior (FIG. 1B) (Neuman-Silberberg and Schupbach, 1996; Roth et al., 1995). During these stages, grk mRNA was below the level of detection by FISH. However, Grk protein was detectable by antibody staining. In armi^{72.1} oocytes, Grk was dispersed throughout the cytoplasm (FIG. 1B). During mid-oogenesis, grk mRNA and Grk protein accumulate at the dorsal-anterior corner of the oocyte (FIG. 1A) (Neuman-Silberberg and Schupbach, 1996; Roth et al., 1995). In armi mutants, grk mRNA was undetectable by FISH. However, using a colorimetric detection method, grk mRNA formed a weak ring near the anterior cortex (FIG. 1A, inset), and immunostaining showed that Grk protein was dispersed throughout the oocyte (not shown). These observations indicated that the armi gene is required for axial polarization of the oocyte during early and mid-oogenesis.

### Example II

### Characterization of Armi Gene

To clone the armi gene, DNA adjacent to the armi¹ P-element insertion was recovered by plasmid rescue. The flanking DNA matched genomic sequences at polytene region 63E1 and the cDNA clone GM10845.5prime in the Berkeley Drosophila Genome Project (BDGP) database (http://www.fruitfly.org). Consistent with a role for armi during oogenesis, GM10845 was isolated from a germarium through stage 6 egg chamber library. GM10845 contained a 2.1 kb insert with 484 nucleotides of putative 5′ UTR upstream of a open reading frame that extended to the end of the clone. Comparison of plasmid rescue, cDNA and genomic sequences indicated that the armi¹ P-element was inserted in the 5′ UTR of the putative transcription unit (see below and FIG. 2).

Northern blots probed with GM10845 detected a primary transcript of approximately 4.2 kb. This transcript was expressed at high levels in ovaries, 0 to 3 hour embryos and in Drosophila DL2 cells, and at low levels at other developmental stages and in ovariectomized females. In some RNA preparations, a minor transcript of roughly 6.9 kb was also detected. The levels of both transcripts were significantly reduced in armi¹ and armi^{72.1} mutant ovaries, and both were restored to wild type levels in armi^{rev39.2} ovaries. Longer exposures revealed low levels of a 4.2 kb transcript and a lower molecular weight transcript in both armi mutants, indicating that both alleles may produce some active protein.

The Northern blot data and DNA sequence analysis indicated that GM10845 is a partial cDNA lacking the 3′ end. To clone a full-length cDNA, 3′-RACE was performed on cDNA prepared from wild type ovary RNA. The recovered 3′ sequences encode an open reading frame terminating in a stop codon followed by a 3′ UTR with a consensus polyadenylation signal and a poly(A) tail. A full-length cDNA was generated by ligation of GM10845 and the 3′ clone. Subsequent genetic tests indicated that the resulting composite cDNA encodes functional Armi protein. Alignment of the composite cDNA sequence with genomic sequence indicated that the mature 4.2 kb mRNA is the product of a 5 kb primary transcript with eight exons (FIG. 2A). Since the 4.2 kb cDNA terminates roughly 500 nucleotides upstream of the cyclin J (cycJ) gene, and the sum of this transcript and the cycJ transcript is roughly 6.2 kb. The larger 6.9 kb transcript was likely to be a read-through product from armi into cycJ. Primers spanning the intergenic region were used to assay for this read-through transcript in RT-PCR reactions (FIG. 2). A fragment consistent with a read-through transcript was obtained. Western blots of wild type and armi¹ ovary extracts were probed with anti-Cyclin J antibody to test whether the armi¹ P-element also disrupted cycJ expression. Similar levels of Cyclin J protein were detected in both samples. Therefore, the observed defects in axial patterning were not due to loss of Cyclin J expression.

To confirm that the 4.2 kb transcript encodes armi, a GFP-armi transgene was used to rescue the chromosomal mutation. GFP was fused to the amino terminus of the 4.2 kb transcript open reading frame, and the fusion was placed under UASp-Gal4 control (Rorth, 1998) (see Experimental Procedures). The resulting fusion construct was introduced into the germline, and crossed into armi mutant backgrounds. Germline expression was induced using the nanos-Gal4 driver (Tracey et al., 2000). Expression of GFP-Armi rescued the female sterility of armi¹ and armi^{72.1} homozygotes and armi^{72.1} hemizygotes. In all three armi backgrounds, transgene expression increased hatch rates from 0% to roughly 60%, indicating that the 4.2 kb transcript encodes functional Armi protein.

Conceptual translation of the open reading frame in the 4.2 kb armi cDNA produced an 1188 amino acid protein of approximately 136 kDa. The first 500 amino acids show no significant homology to known proteins and do not contain conserved structural motifs. The C-terminal domain, however, shows significant homology to a group of putative RNA helicases from plants, mice and humans (FIG. 2B). This domain contains eight motifs characteristic of the Upflp family of ATP-dependent RNA helicases (FIG. 2C); Koonin, 1992; Linder and Daugeron, 2000; Tanner and Linder, 2001; Weng et al., 1996). Armi is most closely related to Mov10 (gb110) proteins in mice and humans (33% identity) (Mooslehner et al., 1991) and to Arabidopsis SDE3 (34% identity) (Dalmay et al., 2001). A C. elegans homolog has not been identified. The function of mammalian Mov10 is not known. SDE3, however, is required for PTGS (Dalmay et al., 2001; Himber et al., 2003), an RNA silencing mechanism related to RNAi in animals (Bernstein et al., 2001; Tijsterman et al., 2002b). Plant PTGS targets viral mRNAs for degradation and thus provides protection against viral infection. This system can also induce degradation of RNAs encoded by transgenes and by homologous endogenous genes (Kooter et al., 1999; Vaucheret and Fagard, 2001). SDE3 also participates in amplification of the silencing trigger, which is required for long-range cell-to-cell spreading of silencing activity (Himber et al., 2003). Propagation of RNA silencing occurs in plants (Palauqui et al., 1997; Voinnet et al., 1998) and nematodes (Fire et al., 1998; Winston et al., 2002), but not in Drosophila (Celotto and Graveley, 2002; Roignant et al., 2003; Schwarz et al., 2002; Tang et al., 2003).

### Example III

### Armi and Components of the RNAi Pathway were Required for Osk mRNA Silencing

The SDE3 homology indicated that armi might function in RNA silencing during oogenesis. Both osk and bcd mRNAs are produced through most of oogenesis, but remain translationally silent until mid-oogenesis or egg activation, respectively (Kim-Ha et al., 1995; Lieberfarb et al., 1996; Markussen et al., 1995; Rongo et al., 1995; Salles et al., 1994). To determine whether armi mutations lead to premature translation of these mRNAs, egg chambers were immunolabeled for Bcd and Osk proteins. Bcd was undetectable during oogenesis in both wild type and armi mutants, indicating that translational repression of bcd did not require armi. Wild type embryos were immunolabeled for Bicoid as a positive control for bcd translation. In wild type egg chambers, Osk protein is not produced until osk mRNA localizes to the posterior pole in mid-oogenesis (Kim-Ha et al., 1995; Lieberfarb et al., 1996; Markussen et al., 1995; Rongo et al., 1995; Salles et al., 1994). However, in armi^{72.1} mutants Osk was produced in early oocytes before transcript localization. Osk started to accumulate as soon as transcript was apparent by FISH. Osk was also prematurely expressed in armi¹ and armi^{72.1} hemizygous oocytes, but was not apparent in early armi^{rev39.2} revertant oocytes.

Late stage egg chambers make up most of the ovary, and Osk is highly expressed during these stages in wild type oocytes. Western blotting was therefore unable to biochemically measure increased Osk protein expression in armi mutants. However, direct cytological comparison of Osk protein labeling in wild type and mutant ovaries was achieved through performance of identical labeling and imaging procedures for all samples. Comparable levels of osk mRNA, as judged by FISH and Northern blotting, were present in armi mutants and in wild type. armi mutants thus do not appear to affect osk transcript stability, but disrupt osk mRNA translational silencing.

In animals, miRNAs repress translation without affecting transcript stability, most likely through direct base pairing with target transcripts (Brennecke et al., 2003; Olsen and Ambros, 1999; Seggerson et al., 2002; Wightman et al., 1993). miRNAs require components of the RNAi pathway for their maturation, and to assemble into the RISC (Caudy et al., 2002; Grishok et al., 2001; Hutvágner et al., 2001; Hutvágner and Zamore, 2002; Ishizuka et al., 2002; Ketting et al., 2001). To determine if components of the RNAi pathway are required for osk mRNA silencing, ovaries mutant for spindle-E (spn-E), aubergine (aub), and maelstrom (mael) were assayed for osk mRNA localization and Osk protein expression. aub and spn-E encode components of the Drosophila RNAi system (Aravin et al., 2001; Kennerdell et al., 2002), and mael is required for localization of a subset of RNAi pathway components in early Drosophila egg chambers (Findley et al., 2003). Results of these experiments are summarized in Table 1:

TABLE 1
Localization of osk mRNA and Osk protein in RNAi mutants

| | wild type | armi | aub | Spn-E | mael |
|---|---|---|---|---|---|
| osk | Posterior | Diffuse | Diffuse | Diffuse | Diffuse |
| mRNA | Cortex | | | | |
| Osk | Not Expressed | Expressed | Expressed | Expressed | Expressed |
| protein | | | | | |

Mutations in aub, Spn-E and mael all led to premature Osk protein expression without dramatically affecting the level of osk mRNA. The amount of Osk protein in aub^{HN2}/aub^{QC42} oocytes was lower than in armi, spn-E, and mael mutants, however neither aub mutation is a null allele, and the level of Osk expression was consistently above the background staining observed in wild type controls. Significantly, mutations in aub, spn-E and mael produced defects in osk mRNA localization during early and mid-oogenesis, as well as defects in D-V patterning that were strikingly similar to those produced by mutations in armi (Clegg et al., 1997; Gillespie and Berg, 1995; Schupbach and Wieschaus, 1991; Wilson et al., 1996). Therefore, multiple components of the Drosophila RNAi system were required for osk mRNA silencing and embryonic axis specification.

### Example IV

### Armi Protein was Asymmetrically Distributed in Early Stage Egg Chambers

To examine the distribution of endogenous Armi protein during oogenesis, polyclonal antobodies against a C-terminal peptide and N-terminal fusion protein were generated (see Experimental Procedures). On Western blots, the affinity purified antibodies reacted with a polypeptide of the molecular weight expected for Armi, and both antibodies strongly labeled germline structures during oogenesis that were absent in armi mutants. These antibodies also produced diffuse labeling in the somatic follicle cells in wild type and armi mutant egg chambers. This may have been non-specific labeling, as whole-mount in situ hybridization showed that armi transcripts were restricted to the germline and clonal analysis indicated that armi function was required in the germline for female fertility and axial patterning. The GFP-Armi fusion protein was incorporated into similar germline structures in living ovarioles, confirming that the antibody labeling was specific.

Armi protein was first apparent early in oogenesis, in the cytoplasm of stem cells and mitotically dividing cystoblasts. In regions 2a and 2b of the germarium, Armi protein was most concentrated in the center of the germline cysts, where the pro-oocyte is located. In stage 1 and early stage 2 egg chambers, Armi accumulated at the anterior of the oocyte, near the ring canals. Armi also extended through the ring canals forming a branched structure that linked the early oocyte with adjacent nurse cells. In stage 3 cysts, Armi accumulated at the posterior cortex, and localized to extensions that pass through the oocyte into the nurse cells.

The distribution of Armi in the germarium was reminiscent of the fusome—a vesiculated structure rich in membrane skeletal proteins that plays a critical role in orienting the cystoblast divisions (Deng and Lin, 1997; Lin et al., 1994). Double immunolabeling for Armi and the fusome marker Adducin, demonstrated that the branched structure formed by Armi assembled as the fusome degenerated. During early oogenesis, Armi protein also accumulated in punctuate structures around the nurse cell nuclei. This distribution was characteristic of nuage—amorphous perinuclear material implicated in RNA processing and transport (Eddy, 1975; Findley et al., 2003; Ikenishi, 1998). Through stages 4 to 7, Armi continued to be somewhat enriched at the posterior cortex of the oocyte, but at significantly lower levels. In stage 9 to 10 egg chambers, Armi was found throughout the cytoplasm of the oocyte and nurse cells, with slight enrichment at the oocyte cortex.

Armi protein and osk mRNA both accumulated in the oocyte during early oogenesis, when armi mutations led to premature Osk protein expression. To define the spatial relationship between Armi protein and osk mRNA, wild type egg chambers were immunostained for Armi and labeled for osk mRNA by FISH. In the germarium, osk mRNA and Armi protein were concentrated in similar regions, but the distributions did not precisely overlap. During stages 3 through 6, Armi protein and osk transcripts were concentrated near the posterior cortex of the oocyte in close proximity, but they assembled into distinct structures. These observations indicated that most of the osk mRNA in the oocyte was not physically associated with Armi protein.

The branched structure formed by Armi in early egg chambers also resembled the polarized microtubule cytoskeleton that directs asymmetric mRNA localization (Theurkauf et al., 1993; Theurkauf et al., 1992). Immunolabeling of wild type ovaries for Armi and α-tubulin demonstrated that Armi protein was closely associated with the microtubule network in early egg chambers. Moreover, disruption of the microtubule network with colchicine disrupted the branched Armi network. However, Armi did not precisely colocalize with microtubules. In stage 1 egg chambers, for example, Armi accumulated near the MTOC, and along microtubules that extended from the MTOC and passed through the ring canals. In single confocal optical sections, the most intense Armi labeling did not overlap with the MTOC or microtubule bundles, which appeared to exclude Armi. Thus, most of the Armi protein did not directly associate with microtubules. Nonetheless, microtubules were required to establish the asymmetric distribution of Armi during early oogenesis.

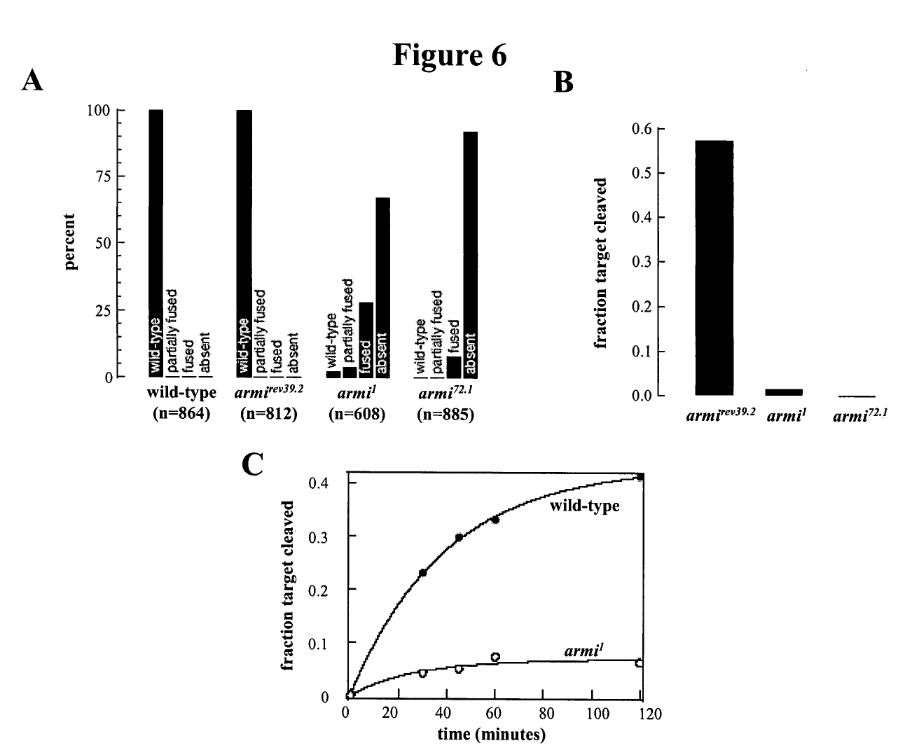

To determine if armi was required for axial polarization of the microtubule cytoskeleton, armi egg chambers were immunolabeled with anti-tubulin antibodies. armi mutations did not affect initial organization of microtubules in stage 1 oocytes. During stages 2 to 7, microtubules were significantly more abundant in the oocyte than in the nurse cells and were organized by the posterior cortex of the oocyte (FIG. 6b). In armi mutants, a posterior MTOC was not apparent and microtubule levels in the oocyte and nurse cells were comparable. Armi was therefore essential for polarization of the oocyte cytoskeleton during early oogenesis. Significantly, spn-E, mael, and aub are also required for microtubule reorganization during early oogenesis (Clegg et al., 2001).

As discussed above, this early polarized microtubule network was required for Grk-dependent differentiation of the posterior follicle cells, which in turn was required for triggering loss of cortical microtubules at the posterior pole during mid-oogenesis. Consistent with the observed defects in microtubule organization and Grk protein localization in early armi oocytes, cortical microtubules persisted at the posterior cortex of armi mutants in mid-oogenesis. Loss of these posterior microtubules was thought to be essential for osk mRNA localization (Cha et al., 2002; Micklem et al., 1997; Shulman et al., 2000). These observations indicated that the RNAi system was required for initial anterior-posterior polarization of the microtubule cytoskeleton, which in turn was required for osk mRNA localization and posterior patterning during mid-oogenesis.

Mutations in armi and the other RNAi components led to premature expression of Osk protein, raising the possibility that Osk misexpression directly or indirectly triggered the defects in microtubule organization. To test this, microtubule organization was analyzed in ovaries overexpressing Osk protein from a transgene (Riechmann et al., 2002). Microtubule organization in these ovaries was indistinguishable from wild type controls. Moreover, armi¹ osk⁵⁴ and armi^{72.1}osk⁵⁴ double mutant ovaries showed microtubule defects that were cytologically identical to the parental armi mutation. Therefore, the defects in osk mRNA silencing and microtubule polarization were genetically distinct consequences, indicating that the RNAi pathway silences osk mRNA and additional transcripts encoding cytoskeletal regulators.

### Example V

### RNAi Pathway Activity in Axis Specification

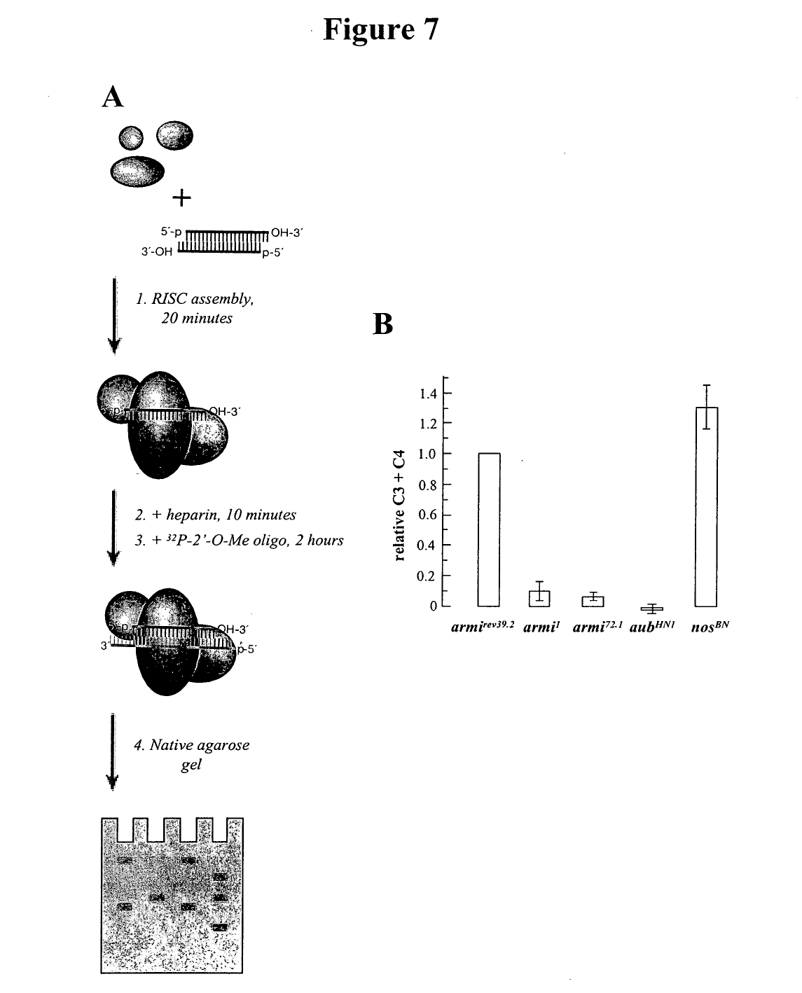

The spectrum of defects observed in RNAi mutations indicated that the RNA silencing machinery targets multiple functions during early oogenesis. The endogenous miRNAs that mediate RNA silencing likely bind complementary sequences in the 3′UTRs of numerous target transcripts, indicating that they coordinate translational control of gene cassettes during complex biological processes (Stark et al., 2003). Interestingly, a computational screen for miRNA targets identified osk mRNA, Kinesin heavy chain mRNA, and transcripts encoding several other cytoskeletal proteins involved in oogenesis as targets for the miR-280 miRNA (Stark et al., 2003; FIG. 7). Kinesin, like the RNAi components, is required for posterior and D/V axis specification (Brendza et al., 2000; Brendza et al., 2002; Duncan and Warrior, 2002; Januschke et al., 2002). This motor also drives ooplasmic streaming during late oogenesis (Palacios and St Johnston, 2002), and mutations in mael lead to premature ooplasmic streaming (Clegg et al., 1997). This could reflect over-expression of Kinesin due to defects in silencing. These observations support the idea that the RNAi system, through miR-280 and other miRNAs, coordinates axis specification by silencing osk mRNA and simultaneously regulating genes involved in microtubule reorganization.

### Example VI

### Armi was Required for Ste Silencing

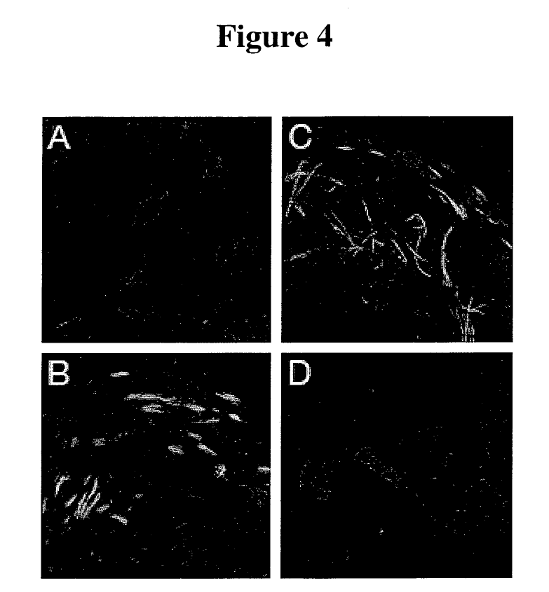

Silencing of the X-linked Ste gene by the highly homologous Y-linked Su(Ste) locus is an example of endogenous RNAi (Aravin et al., 2001; Gvozdev et al., 2003). In Drosophila testes, symmetrical transcription of Su(Ste) produces dsRNA, which is processed into siRNAs (Gvozdev et al., 2003). Su(Ste) siRNAs direct the degradation of Ste mRNA (Aravin et al., 2001). Inappropriate expression of Ste protein in testes is diagnostic of disruption of the RNAi pathway. Both the Argonaute protein, Aubergine, and the putative DEAD box helicase, Spindle-E (Spn-E), are required for RNAi in Drosophila oocytes (Kennerdell et al., 2002). Mutants in the genes encoding either of these proteins fail to silence Ste, as evidenced by the accumulation of Ste protein crystals in testes in aub and spn-E mutants (Schmidt et al., 1999; Stapleton et al., 2001). No Ste protein was detected in wild-type testes (FIG. 4A). Strikingly, Ste protein accumulated in testes of two different armi alleles, armi¹ and armi^{72.1}, (FIG. 4B and C). Neither allele is expected to be a true null, because armi¹ was caused by a P-element insertion 5′ to the open reading frame, whereas armi^{72.1}, which was created by an imprecise excision of the armi¹ P-element, corresponds to a deletion of sequences in the 5′ untranslated region (data not shown). Ste silencing was re-established in males homozygous for the revertant chromosome, armi^{rev39.2} which was generated by excision of the armi¹ P-element (FIG. 4D). These data indicated a role for Armi in Drosophila RNAi.

Immunofluorescent detection of Ste protein in testes implicated both armi alleles in endogenous RNAi, but provided only a qualitative measure of allele strength. Since Ste protein in males reduces their fertility (Belloni et al., 2002), the percent of embryos that hatched when mutant males were mated to wild-type (Oregon R) females provided a more quantitative measure of Ste dysregulation. Hatch rates for the offspring of wild-type, armi¹, armi^{72.1}, and spn-E¹ homozygous males mated to Oregon R females were measured. For spn-E¹ males, 82% of the progeny hatched (n=652). 75% of the progeny of armi¹ males hatched (n=571), but only 45% for armi^{72.1} (n=710). In contrast, 97% of the offspring of wild-type males hatched (n=688). Thus, armi^{72.1} is a stronger allele than armi¹, at least with respect to the requirement for armi in testes.

### Example VII

### Ovary Lysate Recapitulation of RNAi in vitro

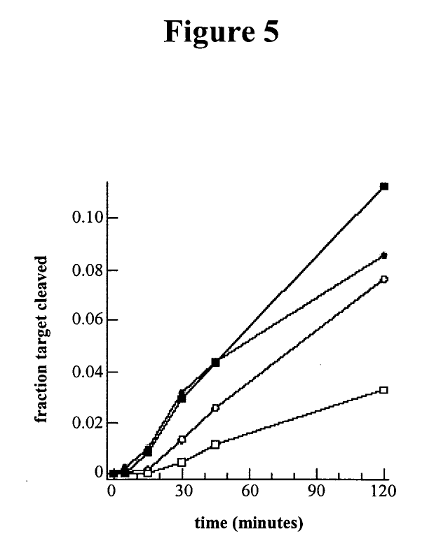

Drosophila syncitial blastoderm embryo lysate has been used widely to study the RNAi pathway (Tuschl et al., 1999). However, armi flies lay few eggs, making it difficult to collect enough embryos to make lysate. To surmount this problem, lysates were prepared from ovaries manually dissected from wild-type or mutant females. Approximately 10 μl of lysate can be prepared from ˜50 ovaries. The well-characterized siRNA-directed mRNA cleavage assay (Elbashir et al., 2001b; Elbashir et al., 2001c) was used to evaluate the capacity of ovary lysate to support RNAi in vitro. Incubation in ovary lysate of a 5′ ³²P-cap-radiolabeled firefly luciferase mRNA target with a complementary siRNA duplex yielded the 5 ′ cleavage product diagnostic of RNAi. siRNAs containing 5′ hydroxyl groups were rapidly phosphorylated in vitro and in vivo, but modifications that block phosphorylation eliminated siRNA activity (Nykänen et al., 2001; Chiu and Rana, 2002; Martinez et al., 2002; Schwarz et al., 2002; Saxena et al., 2003). Replacing the 5′ hydroxyl of the antisense siRNA strand with a 5′ methoxy group completely blocked RNAi in the ovary lysate. In Drosophila, siRNAs bearing a single 2′-deoxy nucleotide at the 5′ end were poor substrates for the kinase that phosphorylates 5′ hydroxy siRNAs (Nykänen et al., 2001). A comparison of initial cleavage rates showed that in ovary lysate, target cleavage was slower for siRNAs with a 2′-deoxy nucleotide at the 5′ end of the anti-sense strand than for standard siRNAs (FIG. 5). Furthermore, the rate of target cleavage was fastest when the siRNA was phosphorylated before its addition to the reaction (FIG. 5). A similar enhancement from pre-phosphorylation was reported for siRNA injected into Drosophila embryos (Boutla et al., 2001). It was therefore concluded that lysates from Drosophila ovaries faithfully recapitulate RNAi directed by siRNA duplexes.

### Example VIII

### Armi Ovary Lysates Exhibited RNAi Defects

In contrast to wild-type, lysates prepared from armi^{72.1} ovaries did not support siRNA-directed target cleavage in vitro: no cleavage product was observed in the armi^{72.1} lysate after 2 hours. This result was observed for at least 10 independently prepared lysates. To determine if the RNAi defect was allele specific, ovaries from armi¹ were examined. Phenotypically, this allele is weaker than armi^{72.1} in its effects on both male fertility (above) and oogenesis. For armi^{72.1} females, 92% of the eggs lacked dorsal appendages, compared to 67% of armi¹ eggs, and some armil eggs had wild-type or partially fused dorsal appendages (FIG. 6A). Consistent with its weaker phenotype, the armi¹ allele showed a small amount of RNAi activity in vitro (FIG. 6B). The two alleles were analyzed together at least four times using independently prepared lysates. In all assays, total protein concentration was adjusted to be equal. Lysate from the revertant allele, armi^{rev39.2} which had wild-type dorsal appendages, showed robust RNAi, demonstrating that the RNAi defect in the mutants is caused by mutation of armi, not an unlinked gene.

### Example IX

### Impaired RISC Assembly Observed in Armi Ovary Lysates

The rate of target cleavage was much slower for armi¹ than for wild-type (FIG. 6C). Since the rate of target cleavage in this assay usually reflects the concentration of RISC (Schwarz et al., in press), it was proposed that armi mutants were defective in RISC assembly. To test this hypothesis, a new method to measure RISC was developed that requires less lysate than previously described techniques (FIG. 7A). Double-stranded siRNA was incubated with ovary lysate in a standard RNAi reaction. To detect RISC, a 5′-³²P-radiolabeled, 2′-O-methyl RNA oligonucleotide complementary to the anti-sense strand of siRNA was added. Like target RNAs, 2′-O-methyl oligonucleotides bind to RISC containing a complementary siRNA, but unlike RNA targets, they cannot be cleaved and binding is essentially irreversible (Hutvágner et al., 2004). RISC/2′-O-methyl oligonucleotide complexes were then resolved by electrophoresis through an agarose gel. After the 20 minute incubation, but before the addition of the 2′-O-methyl oligonucleotide, heparin was added to reduce non-specific binding of proteins to the 2′-O-methyl oligonucleotide and to quench RISC assembly. Mature RISC was refractory to heparin: 1 mg/ml (final concentration) heparin added at the start of the reaction blocked RNAi, but had no effect when added after RISC assembly but before the target RNA.

To validate the method, RISC formation was first examined in embryo lysate. Four distinct complexes (C1, C2, C3, C4) were formed when siRNA was added to the reaction. Formation of the complexes required ATP and was disrupted by pre-treatment of the lysate with the alkylating agent N-ethylmaleimide (NEM), but refractory to NEM-treatment after RISC assembly, all properties of RNAi itself (Nykänen et al., 2001). No complex was observed when the siRNA was unrelated to the 2′-O-methyl oligonucleotide. The amount of complex formed by different siRNA sequences correlated well with their capacity to mediate cleavage (data not shown). The four complexes were also detected in wild-type ovary lysate, indicating that the same RNAi machinery is used during oogenesis and early embryogenesis. A lower amount of RISC was formed in ovary compared to embryo lysates, an outcome explained by the lower overall protein concentration of ovary lysates.

The 2′-O-methyl oligonucleotide/native gel assay was used to analyze RISC assembly in armi mutant ovary lysates. armi mutants were deficient in RISC assembly. Formation of C3/C4 RISC complex was particularly robust in both wild-type and armi^{rev39.2} siRNA-treated ovary lysates, while dramatically less C3/C4 complex was observed in armi¹, armi^{72.1} and aub^{HN2} siRNA-treated ovary lysates (quantitative results from four independent assays are shown in FIG. 7B). The extent of the RISC assembly deficit correlated with allele strength: less C3/C4 complex formed in lysate from the strong armi^{72.1} allele than from armi¹ (FIG. 7B). Compared to the phenotypically wild-type armi^{rev39.2}, >10-fold less RISC was produced in armi^{72.1}. The defect in RISC assembly in armi mutants was similar to that observed in lysates from aub^{HN2} ovaries (data not shown). aub mutants do not support RNAi following egg activation and fail to silence the Ste locus in testes (Schmidt et al., 1999; Kennerdell et al., 2002), and lysates from aub^{HN2} ovaries do not support RNAi in vitro (data not shown). Aub is one of five Drosophila Argonaute proteins, core constituents of RISC. It was therefore not surprising that Aub was required for RISC assembly. Since RISC assembly in vitro was not detectable in aub^{HN2} lysates, these data indicated that Aub is the primary Argonaute protein recruited to exogenous siRNA in Drosophila ovaries. In contrast, ovaries from nanos^{BN}, a maternal effect mutant not implicated in RNAi, were fully competent for both RISC assembly (FIG. 7B) and siRNA-directed target RNA cleavage (data not shown).

### Example X

### Identification of Intermediates in RISC Assembly

These data indicated that both armi and aub are required genetically for RISC assembly, but they provided no insight into the molecular basis of their RISC assembly defect(s). To define the step(s) in RISC assembly at which armi and aub mutants are blocked, protein-siRNA intermediates in the RISC assembly pathway were identified. The native gel assay employed above relied upon detection of a radiolabeled 2′-O-methyl oligonucleotide to indicate formation of only those RNA silencing complexes competent to bind target RNA (mature RISC). To observe intermediates in the assembly of RISC, a modified native gel assay—one reliant upon detection of radiolabeled siRNA, rather than radiolabeled 2′-O-methyl oligonucleotide—was designed. The radiolabeled siRNA of the modified native gel assay allowed for detection of complexes containing either single-stranded or double-stranded siRNA. Through use of functionally asymmetric siRNAs (Schwarz et al., 2003), complexes containing single- and double-stranded siRNA were readily distinguished.

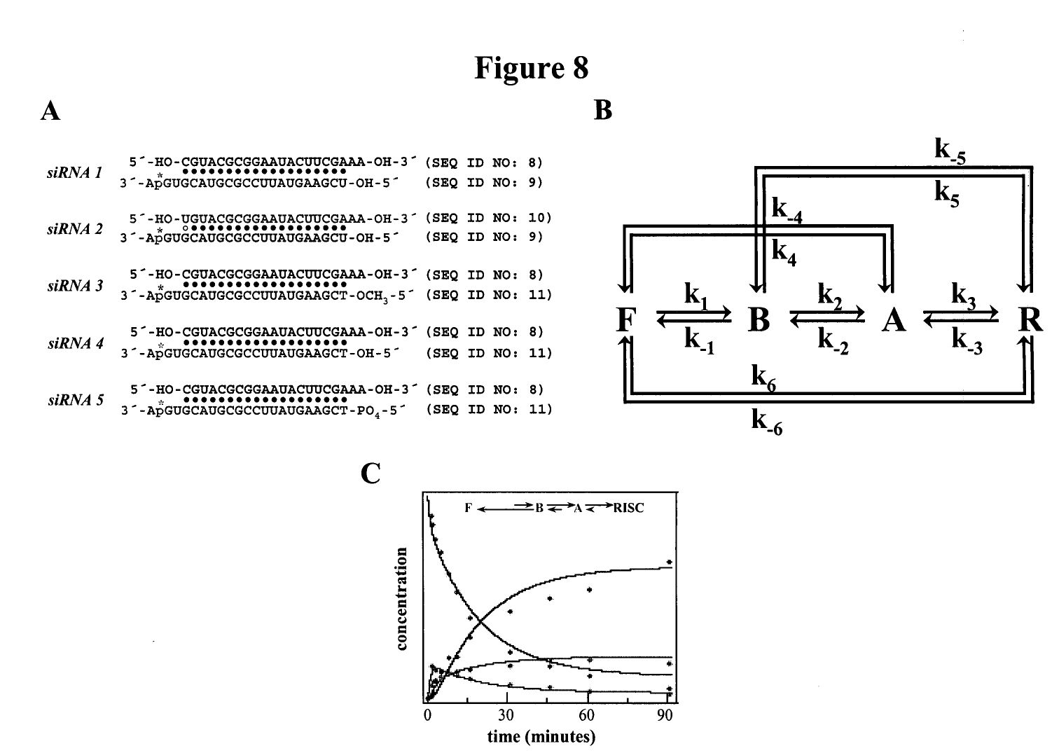

RISC contains only a single siRNA strand (Martinez et al., 2002; Schwarz et al., 2002; Schwarz et al., 2003). Functionally asymmetric siRNAs load only one of the two strands of an siRNA duplex into RISC and degrade the other strand (Schwarz et al., 2003); the relative stability of the 5′ ends of the two strands determines which is loaded into RISC (Aza-Blanc et al., 2003; Khvorova et al., 2003; Schwarz et al., 2003). siRNA 1 (FIG. 8A), loads its anti-sense strand into RISC, whereas siRNA 2 loads the sense strand (Schwarz et al., 2003). The two siRNA duplexes are identical, except that siRNA 2 contains a C-to-U substitution at position 1, which inverts the asymmetry (Schwarz et al., 2003). For both siRNAs, the anti-sense strand was 3′ ³²P-radiolabeled and would always be present in complexes that contain double-stranded siRNA. However, RISC would contain the ³²P-radiolabeled anti-sense strand only for siRNA 1. siRNA 2 would also make RISC, but it would contain the non-radioactive sense strand.

When either siRNA 1 or siRNA 2 was used to assemble RISC in embryo lysate, two distinct complexes (B and A, with complex B exhibiting higher mobility than complex A; data not shown) were detected in the native gel assay; a third distinct complex, of lower mobility than either complex B or A, was detected only with siRNA 1. This third complex therefore contained single-stranded siRNA and corresponded to RISC. Complexes B and A were good candidates for RISC assembly intermediates. Formation of all three complexes was dramatically reduced when the anti-sense siRNA strand contained a 5′ methoxy group (siRNA 3), a modification which blocks RNAi (Nykänen et al., 2001). When the anti-sense strand of the siRNA contained a single 5′ deoxynucleotide, making it a poor substrate for phosphorylation in the lysate (Nykänen et al., 2001), assembly of all three complexes was reduced (siRNA 4). Phosphorylating the 5′ deoxysubstituted siRNA before the reaction restored complex assembly (siRNA 5). Formation of complex A and of RISC required ATP. In contrast, complex B assembled efficiently in the absence of ATP, but only if the siRNA was phosphorylated prior to the reaction.

Complexes B, A, and RISC also formed in ovary lysate. As observed for embryo lysate, complexes B and A contained double-stranded siRNA, whereas RISC contained only single-stranded. No complexes formed in ovary lysate when siRNA 5′ phosphorylation was blocked (siRNA 3) and complex assembly was reduced when siRNA phosphorylation was slow (siRNA 4).

To determine the relationship of complexes B, A, and RISC, the kinetics of complex formation were monitored and data were analyzed by kinetic modeling (FIG. 8B, C). Of all possible models relating free siRNA, B, A, and RISC, only the simple linear pathway siRNA→B→A→RISC fit well to these data (see Experimental Procedures). The modeled rate constants for the pathway were consistent with the observation that formation of complex B was ATP-independent, but RISC formation was ATP-dependent.

A ‘chase’ experiment was also performed to confirm that complex B constituted a precursor to RISC (via a complex A intermediate). Complex B was assembled by incubating ³²P-radiolabeled siRNA in embryo lysate for 5 minutes, then a 20-fold excess of unlabeled siRNA was added to prevent further incorporation of ³²P-siRNA into complex. The incubation was then continued and formation of complexes was monitored. Complex B disappeared with time, while complex A increased with time, then peaked at ˜60 minutes. RISC accumulated throughout the experiment. The amount of radiolabeled free siRNA was essentially unchanged throughout the experiment, demonstrating that the unlabeled siRNA effectively blocked incorporation of ³²P-free siRNA into complex. Thus, complex B was chased into RISC, likely via complex A. Together, the kinetic modeling and ‘chase’ experiment data provide support for a RISC assembly pathway in which the siRNA passes through two successive, double-stranded siRNA-containing complexes, B and A, in order to be transformed into the single-stranded siRNA-containing RISC.

### Example XI

### Complex A Contains the R2D2/Dicer-2 Heterodimer

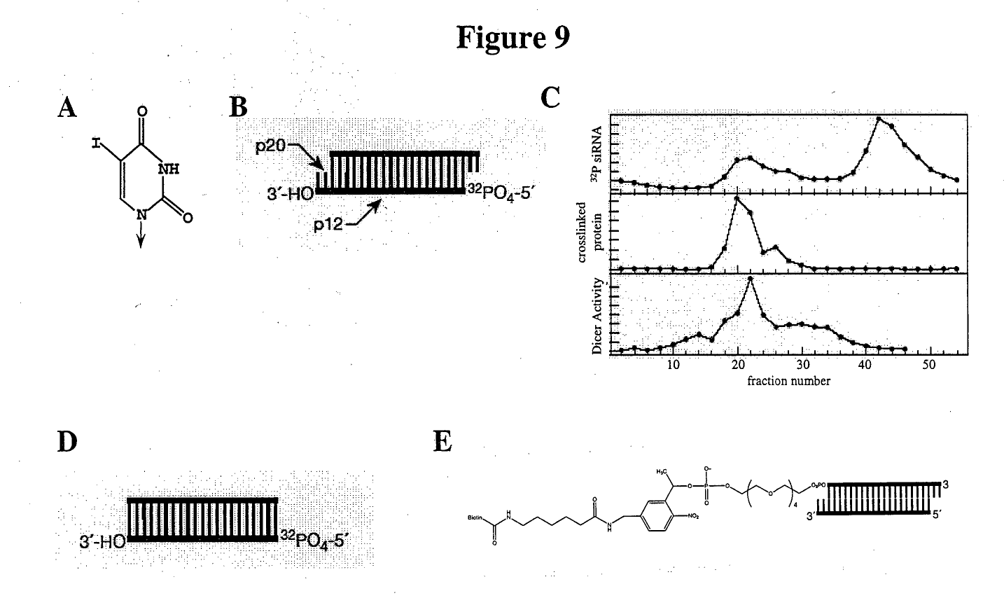

Liu and colleagues previously proposed that a heterodimeric complex, comprising Dicer-2 (Dcr-2) and the double-stranded RNA-binding protein R2D2, loads siRNA into RISC (Liu et al., 2003). Complex A contained the Dcr-2/R2D2 heterodimer. R2D2 and Dcr-2 were readily crosslinked to ³²P-radiolabeled siRNA with UV light (Liu et al., 2003). An siRNA was synthesized containing a single photocrosslinkable nucleoside base (5-iodouracil) at position 20 (FIG. 9A, B). The ³²P-5-iodouracil siRNA was incubated with embryo lysate to assemble complexes, and was then irradiated with 302 nm light, which initiated protein-RNA crosslinking only at the 5-iodo-substituted nucleoside. Proteins covalently linked to the ³²P-radiolabeled siRNA were resolved by SDS-PAGE. Two proteins—˜200 kDa and ˜40 kDa—efficiently crosslinked to the siRNA. Both crosslinked proteins were co-immunoprecipitated with either α-Dcr-2 or α-R2D2 serum, but not normal rabbit serum. Neither crosslink was observed in ovary lysates prepared from R2D2 homozygous mutant females, a result expected because Dcr-2 is unstable in the absence of R2D2 (Liu et al., 2003).

In a related experiment, crosslinkable ³²P-5-iodouracil siRNA was incubated with embryo lysate, and the siRNA-containing lysate was then fractionated by gel filtration chromatography. Chromatographic fractions were exposed to 302 nm UV light to initiate crosslinking. As above, two proteins, ˜200 kDa and ˜40 kDa, were crosslinked to the siRNA, and co-eluted during gel filtration as a single, ˜350 kDa peak (FIG. 9C and data not shown). Efficient crosslinking to the ˜200 kDa protein required both UV-irradiation and the 5-iodo-uracil substitution, did not occur with a blunt-ended RNA duplex (FIG. 9D and data not shown) or single-stranded siRNA (data not shown), and required the 5′ phosphate of the siRNA (data not shown). The crosslinked protein was not detected when the 5-iodouracil was at position 12 of the siRNA (FIG. 9B and data not shown). The apparent molecular weights of the two Drosophila Dicer proteins are ˜210 kDa (Dicer-1; Dcr-1) and ˜190 kDa (Dicer-2; Dcr-2)(Liu et al., 2003). Consistent with the idea that the crosslinked protein was one of the two Drosophila Dicer proteins, Dicer activity—monitored by the conversion of long dsRNA into siRNA—co-purified with the crosslinked proteins (FIG. 9C and data not shown). The results of the crosslinking experiments indicated that one of the two Drosophila Dicer proteins bound tightly to siRNA. This conclusion agreed with earlier studies demonstrating that siRNA duplexes were competitive inhibitors of Dicer activity in Drosophila embryo lysate (Tang et al., 2003). To determine if one or both of the Drosophila Dicer proteins bound tightly to siRNA, an siRNA duplex tethered to paramagnetic beads was used to purify siRNA-bound proteins. Because the siRNA duplex was linked to the beads via the 5′ end of the strand that was not loaded into RISC (FIG. 8A; Schwarz et al., 2003), RISC did not form on the beads (data not shown). Consequently, the tethered siRNA duplex bound proteins that interact with double-stranded, but not single-stranded, siRNA. Proteins bound to the siRNA were recovered by irradiating the beads with 302 nm light to break a photocleavable linkage tethering the siRNA duplex to the beads (FIG. 9E). The only high molecular weight protein liberated by photocleavage was a ˜200 kDa protein. Mass spectrometry of 13 tryptic peptides of this protein revealed the following sequences:

TABLE 2
Peptide-mass fingerprinting of ˜200 kDa Protein (Dicer-2)

| | | | Amino Acid |
|---|---|---|---|
| | | | Position |
| m/z | MH+ | Peptide | in Dicer-2 |
| submitted | matched | Sequences | (SEQ ID NO: 12) |
| 862.49 | 862.5151 | NYAILLR | 753-759 |
| 1212.69 | 1212.6377 | TIQQIYQYR | 514-522 |
| 1225.68 | 1261.6727 | FVLFTADKER | 502-511 |
| 1261.69 | 1348.6398 | NVLTPQFMVGR | 417-427 |
| 1348.69 | 1363.7197 | FVNFQESQGHR | 1539-1549 |
| 1363.69 | 1519.7393 | MYFLLHAEALR | 999-1009 |
| 1519.81 | 1635.8131 | NNISPDFESVLER | 428-440 |
| 1635.86 | 1648.7654 | DLTEQLTFVVHNR | 944-956 |
| | | | (SEQ ID NO: 25) |
| 1648.82 | 1648.7654 | NQFHMPTGNIYGNR | 1135-1148 |
| 1727.87 | 1727.8029 | VGFYVGEQGVDDWTR | 85-99 |
| 1774 | 1773.9288 | YLLQALTHPSYPTNR | 1449-1463 |

The ˜200 kDa protein was therefore exclusively Dcr-2; no Dcr-1 peptides were detected. These data, together with results of the immunoprecipitation experiments described above indicated that the ˜200 kDa protein detected by UV crosslinking was Dcr-2.

To resolve components of complexes B, A and RISC, the radiolabeled siRNA crosslinking assay was repeated, with the reaction analyzed by native gel electrophoresis. Each of complexes B, A and RISC was eluted from the gel and analyzed by SDS-PAGE. R2D2 and Dcr-2 crosslinks were observed to be present in complexes A and RISC, but not B. In a parallel experiment, complexes B, A, and RISC were isolated (without crosslinking), and analyzed by Western blotting with either α-Dcr-2 or α-R2D2 antibodies. Again, complexes A and RISC, but not B, contained both Dcr-2 and R2D2. Finally, complex assembly was tested in ovary lysates prepared from R2D2 homozygous mutant females. Only complex B formed in these lysates. These data demonstrated that complex A contains the previously identified Dcr-2/R2D2 heterodimer (Liu et al., 2003), and that both Dcr-2 and R2D2 remain associated with at least a subpopulation of RISC, consistent with earlier reports that Dcr-2 in flies and both DCR-1 and the nematode homolog of R2D2, RDE-4, co-immunoprecipitate with Argonaute proteins (Hammond et al., 2001; Tabara et al., 2002).

### Example XII

### Armi Mutants are Defective for the Conversion of Complex A to RISC

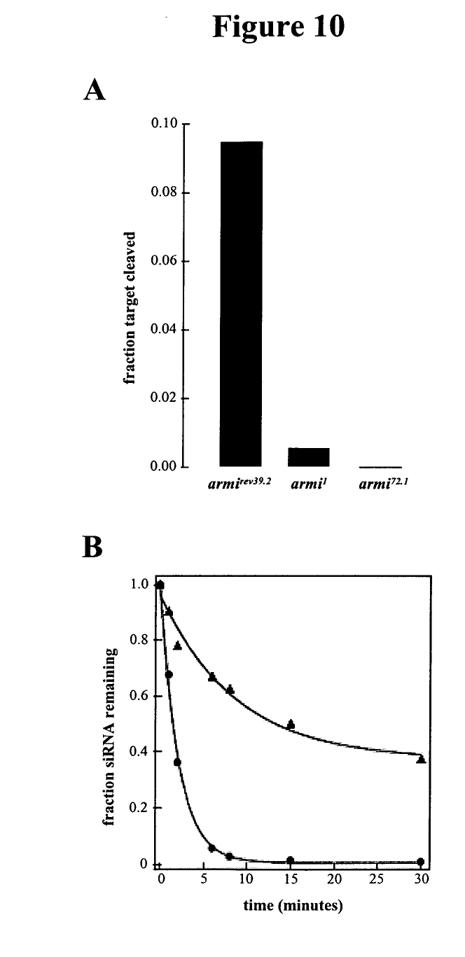

RISC does not form in ovary lysates from armi or aub mutants (FIG. 7B). However, both complex B and A were readily detected in armi and aub mutants. Thus, armi and aub mutants are impaired in a step in RISC assembly after binding of the siRNA to the Dcr-2/R2D2 heterodimer. To investigate whether Armi acts after the formation of complex A to unwind siRNA duplexes prior to their assembly into RISC, single-stranded siRNA was tested for the ability to circumvent the requirement for armi. In vitro and in vivo, single-stranded siRNA triggers RNAi, albeit inefficiently (Martinez et al., 2002; Schwarz et al., 2002). armi ovary lysates failed to support RNAi when the reactions were programmed with 5′-phosphorylated, single-stranded siRNA (FIG. 10A). The defect with single-stranded siRNA correlated with allele strength: some activity was seen in lysates from the weak allele, armi¹, but none for the strong allele, armi^{72.1}. The requirement for a putative ATPase—Armi—in RNAi triggered by single-stranded siRNA indicated the presence of an additional ATP-dependent step in the RISC assembly, after siRNA unwinding. To test if loading of single-stranded siRNA into RISC requires ATP, 5′ phosphorylated, single-stranded siRNA was added to embryo lysates depleted of ATP. After incubation for 2 hours, no cleavage product was detected, indicating that there is at least one ATP-dependent step downstream of siRNA unwinding. The stability of single-stranded siRNA was not reduced by ATP depletion. In fact, single-stranded siRNA was slightly more stable in the absence of ATP (FIG. 10B). Thus differential stability cannot account for the requirement for ATP in RNAi triggered by single-stranded siRNA. In the RNAi pathway, there are at least three steps after siRNA unwinding: RISC assembly, target recognition, and target cleavage. To assess if either target recognition or cleavage was ATP-dependent, single-stranded siRNA was incubated in a standard RNAi reaction with ATP to assemble RISC. Next, NEM was added to inactivate the ATP-regenerating enzyme, creatine kinase, and to block further RISC assembly. NEM was quenched with dithiothreitol (DTT), and hexokinase and glucose were added to deplete ATP. Finally, mRNA target was added and the reaction incubated for 2 hours. Using this protocol, high ATP levels were maintained during RISC assembly, but less than 100 nM ATP was present during the encounter of RISC with the target RNA. Target recognition and cleavage did not require ATP when RISC was programmed with either double- or single-stranded siRNA, provided that ATP was supplied during RISC assembly.

### Example XIII

### A Model for RNA Silencing in Drosophila

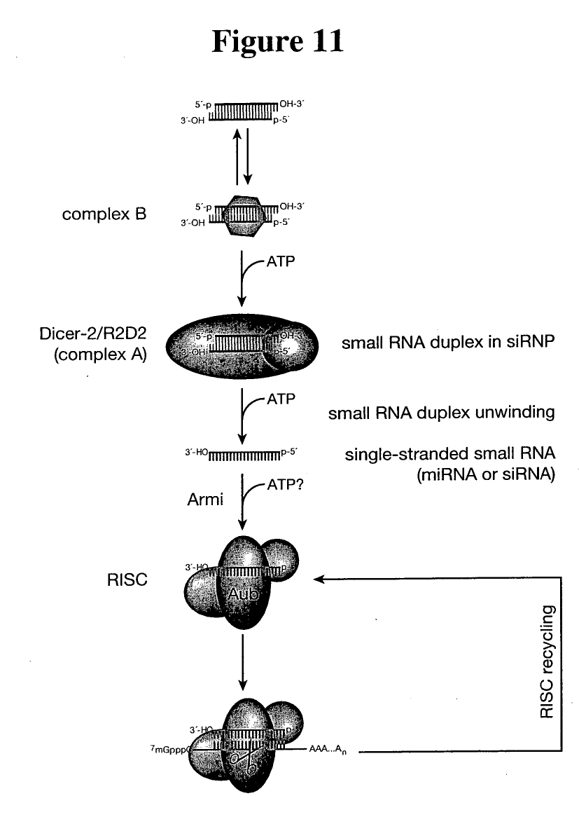

The above experiments identified, for the first time, two intermediates in RISC assembly, termed complex A and complex B. Complex B forms rapidly upon incubation of siRNA in lysate, in the absence of ATP. The siRNA is then transferred to complex A, which contains the previously identified R2D2/Dcr-2 heterodimer. The siRNA is double-stranded in both B and A. RISC is formed from complex A by a process that requires both siRNA unwinding and ATP. Both aub and armi are required genetically for the production of RISC from complex A. The involvement of Armi, a putative RNA helicase protein, in the production of RISC from complex A and the present finding that incorporation of single-stranded siRNA into RISC requires ATP indicate that Armi functions to incorporate single-stranded siRNA into RISC (FIG. 11).

### Example XIV

### Armi Modulatory Compound Screening Assay

In an exemplary assay, identical Armi-expressing cells (e.g. wild-type cells, mutant cells, cells transgenic for armi, or cells induced to express Armi), possibly also containing or expressing siRNA, are grown in microwells, then lysed within microwells and contacted by a library of compounds, such that each cell of a microwell is exposed to a subset of library compounds. A detectably labeled reporter RNA (and possibly a test siRNA) is added to all wells, and the cleavage state of the reporter RNA is ascertained following an appropriate period of incubation. Compounds added to wells that have failed to cleave the reporter RNA are identified as candidate compounds for Armi activity modulation. These candidate compounds are then subjected to additional tests (possibly including performance of further rounds of the initial screening assay) to verify the compound(s)' effects on Armi activity and RNAi pathway activity.

Experimental Procedures

A. General Methods

Target RNA cleavage assay was performed as described (Haley et al., 2003). ATP depletion and NEM quenching were as published (Nykänen et al., 2001).

B. Drosophila Stocks and Genetics

Oregon R, yw and armi^{rev} were used as controls. The armi^{72.1} and armi^{rev} alleles were produced by transposase (Δ2-3)-mediated excision of the P-element insertion in armi¹. Germline clones were generated using the FLP-DFS technique adapted for the third chromosome (Chou and Perrimon, 1996; Theodosiou and Xu, 1998). Mutant alleles and allelic combinations used in this study were aub^{Q42}/aub^{HN2} (Schupbach and Wieschaus, 1991), mael^{r20}/Df(3L)HD1(Clegg et al., 1997), and spn-E¹/spn-E¹ (Gillespie and Berg, 1995; Gonzalez-Reyes et al., 1997). Fly lines for germline clone analysis, aub^{QC42}, aub^{HN2}, Df(3L)E1, Df(3L)HD1 and spn-E¹, were provided by the Bloomington Drosophila Stock Center (Consortium, 2003) http://flybase.org/.

C. Fluorescent In Situ Hybridization

Fluorescent RNA probes were synthesized from bicoid cDNA, pbcdFS (Salles et al., 1994), osk cDNA pBlueosk, (Ephrussi et al., 1991), and gurken 1.7 cDNA (Neuman-Silberberg and Schupbach, 1993) as described (Cha et al., 2001). In situ hybridizations were performed as previously described, omitting enhancement with Alexa488-conjugated anti-fluorescein IgG (Cha et al., 2001). Samples were mounted in PBS, 90% glycerol and analyzed by confocal microscopy using a Leica TCS-SP inverted laser scanning microscope. Identical probes, hybridization and imaging conditions were used for wild type and mutant egg chambers.

D. Cloning and Molecular Characterization of Armi

Genomic sequences flanking the armi¹ P-element insertion were recovered by plasmid rescue (Hamilton and Zinn, 1994). Candidate armi cDNAs GM10845.5prime and GM12116.5prime were cloned and sequenced by the Berkeley Drosophila Genome Project and obtained from Research Genetics. GM12116.5prime did not contain a DNA insert. The SMART RACE cDNA amplification kit (Clontech) was used for 3′-RACE with the following gene specific primer: armi1728R,

CCGTTTCAGATTGCTCCTACACCT (SEQ ID NO: 13)

and for RT-PCR using primer pair: armi330R,

GCTTAAGGCGAATCCAAACGAAAT (SEQ ID NO: 14)

and armi2457L,

TACGAAACGAGGCGAATGAAATCT. (SEQ ID NO: 15)

cDNA fragments were cloned into pBluescript II KS (Stratagene) (details provided upon request), and DNA inserts in three independent clones were sequenced. Northern analysis was performed using the DIG/Genius System (Roche) following manufacturer's recommendations. Primers used for RT-PCR amplification of the armi-cycJ intergenic region were: armi2709,

GGAATCTGCCTGGTGCCCGAATCTT (SEQ ID NO: 16)

and cycJ4109,

GCTCCCTCATGGTCAAAAATATATC. (SEQ ID NO: 17)

The EGFP-Armi germline expressed transgene was constructed in pUASP (Rorth, 1998) adapted for the Gateway System (Invitrogen). Germline transformation was as described (O'Connor and Chia, 1993).
E. Antibody Production and Purification

The rabbit anti-Armi-CT antibody was raised against the C-terminal peptide CLETFVPSLNTTDDLN (SEQ ID NO: 18) coupled to KLH (Peptide Core Facility, University of Massachusetts Medical Center). Antigen injection into rabbits and serum production (Covance Inc) were performed using standard procedures. Armi-CT antisera were affinity purified over Armi-CT peptide coupled to epoxy-activated Sepharose 6B (Pharmacia) as described (Harlow and Lane, 1999). For the anti-Armi-NT1 antibody, amino acids L43 to A445 were ligated to the 6× His tag in the pQE31 (Qiagen), expressed in E. coli strain BL21(DE3). The fusion protein was purified on Probond Ni matrix under denaturing conditions (Invitrogen) and used to immunize rabbits (ResGen, Invitrogen). NT1 antisera was affinity purified on fusion protein coupled to CNBr activated Sepharose 4B (Pharmacia) as described elsewhere (Harlow and Lane, 1999).

F. Immunohistochemistry

Egg chamber fixation and whole-mount antibody labeling were performed as described (Theurkauf, 1994). Armi-CT antibody was used at 1:200 to 1:500 dilutions and Armi-NT1 at 1:500. Microtubules were labeled with FITC-conjugated mouse monoclonal anti-α-tubulin (Sigma Chemical Co) used at 1:200. Oskar protein was labeled with rabbit polyclonal anti-Oskar antibody (Vanzo and Ephrussi, 2002) at 1:2500, Vasa with rabbit polyclonal anti-Vasa antibody (Liang et al., 1994) at 1:1000, Cyclin J with rabbit anti-Cyclin J antibody at 1:2000 (Kolonin and Finley, 2000) and Bicoid protein with rabbit polyclonal anti-Bicoid antibody at 1:50 (Struhl et al., 1989). Monoclonal anti-Gurken monoclonal anti-Adducin antobodes were used at 1:10 and were obtained from the Developmental Studies Hybridoma Bank. Actin was visualized with rhodamineconjugated phalloidin, which as used at 1:100 dilution (Molecular Probes). For microtubule disruption, Oregon R female flies were starved 5 hours and then fed yeast paste with 50 mg/ml colchicine overnight. Oskar protein was misexpressed during early oogenesis using an osk-K10 transgene under UASp-Gal4 control (Riechmann et al., 2002).

G. Stellate Immunofluorescence

Testes were dissected in testes fixation buffer (1 mM EDTA, 183 mM KCl, 47 mM NaCl, 10 mM Tris, pH 6.8) and fixed with formaldehyde as described (Theurkauf, 1994). Ste protein was labeled with anti-Ste IgG at 1:100. Images were analyzed by confocal microscopy using a Leica TCS-SP inverted laser scanning microscope. DNA was stained with TOTO-3 (Molecular Probes).

H. Ovary Lysate Preparation

Wild-type or mutant fly ovaries were dissected with forceps (World Precision Instruments 500232) and collected in 1× PBS buffer in 1.5 ml microcentrifuge tubes. Ovaries were centrifuged at 11,000×g for 5 minutes at 4° C. The PBS was removed from the ovary pellet, then ovaries were homogenized in 1 ml ice-cold lysis buffer (100 mM potassium acetate, 30 mM HEPES-KOH at pH 7.4, 2 mM magnesium acetate) containing 5 mM DTT and 1 mg/ml complete ‘mini’ EDTA-free protease inhibitor tablets (Roche) per gram of ovaries using a plastic ‘pellet pestle’ (Kontes). Lysate was clarified by centrifugation at 14,000×g for 25 minutes at 4° C. The supernatant was aliquoted into chilled microcentrifuge tubes, flash frozen in liquid nitrogen, and stored at −80° C. RNAi reactions were assembled using equal amounts of total protein for all genotypes within an experiment.

I. Synthetic siRNA

The siRNAs were prepared from synthetic 21 nt RNAs (Dharmacon Research). Sense siRNA sequences used were

(SEQ ID NO: 8)

5′-HO-CGU ACG CGG AAU ACU UCG AAA-3′
(5′OH (riboU), 5′CH₃O (2′dT), 5′OH (2′dT))
and

(SEQ ID NO: 19)

5′-HO-UGA GGU AGU AGG UUG UAU AGU-3′(un).

Anti-sense siRNA sequences used were

(SEQ ID NO:20)

5′-HO-UCG AAG UAU UCC GCG UAC GUG-3′
(5′OH (riboU));

(SEQ ID NO:21)

5′-CH₃O-dTCG AAG UAU UCC GCG UAC GUG-3′
(5′CH₃O (2′dT));

(SEQ ID NO:22)

5′-HO-dTCG AAG UAU UCC GCG UAC GUG-3′
(5′OH (2′dT));
and

(SEQ ID NO:23)

5′-HO-UAU ACA ACC UAC UAC CUC AUU-3′(un).

Appropriate pairs of siRNA strands were annealed to form siRNA duplexes as described (Elbashir et al., 2001b) and used at a final concentration of ≦20 nM (FIG. 5) or ≦50 nM (FIG. 6). siRNA single strands were phosphorylated with polynucleotide kinase according to the manufacturer's protocol (PNK; New England Biolabs) and used at 200 nM (final concentration).
J. RISC Assembly

RISC assembly was as described (Zamore et al., 2000), except that the reaction contained 40% (v/v) embryo or ovary lysate and 50 nM siRNA duplex. Lysates were adjusted with lysis buffer to contain equal amounts of protein. Following incubation at 25° C. for 20 minutes, 1 mg/ml heparin was added and incubated for 10 minutes. Heparin served to reduce non-specific binding of proteins to the 2′-O-methyl oligonucleotide and to quench RISC assembly. (Mature RISC is refractory to heparin: 1 mg/ml (final concentration) heparin added at the start of the reaction blocked RNAi in the cleavage assay, but had no effect when added together with target RNA.) Then the 5′-³²P-radiolabeled, 2′-O-methyl oligonucleotide

(SEQ ID NO:24)

(5′-CAU CAC GUA CGC GGA AUA CUU CGA AAU GUC C-3′;)

was added at 2 nM final concentration and incubated for 2 hours. After the addition of 3.0% (w/v) Ficoll-400, complexes were resolved by native gel electrophoresis at 4 Watts for 2 hours at room temperature. Native gels were 1 mm thick, 1.5% (w/v) agarose (GTG grade), 0.5× TBE with 1.5 mM MgCl₂, cast vertically between a standard glass plate and a ground glass plate (National Glass Works, Worcester, Mass.). To detect intermediates in RISC assembly, ³²P-radiolabeled siRNA was incubated with lysate for 1 hour, unless otherwise noted. No heparin was added to these reactions. After incubation, the samples were adjusted to 3.0% (w/v) Ficoll-400 and resolved by vertical native gel electrophoresis as above. Gels were dried under vacuum onto Hybond-N⁺ nylon membrane (Amersham).
K. Kinetic Modeling

Data from native gel analysis of siRNA-containing complexes were initially fit using Berkeley Madonna 8.0.1 software to the global model shown in FIG. 8B. Rates for k₄, k₄, k₅, k₋₅, k₆, and k₋₆ ranged from 5-fold (k₅) to 108-fold (k₋₄) slower than the slowest forward rate for the linear pathway F→B→A→RISC. The data were therefore modeled neglecting rates k₄, k₋₄, k₅, k₋₅, k₆, and k₋₆ to generate FIG. 8C.

L. Crosslinking

5′ ³²P-radiolabeled siRNA duplex was used at 4 million counts per minute in a standard RNAi reaction, incubated 45-60 minutes at 25° C., then transferred to a 96-well round bottom plate on ice. Samples were irradiated for 10-15 minutes with 302 nm light using an Ultraviolet Products model TM-36 transilluminator inverted directly onto the polystyrene lid of the 96-well plate. Samples were then adjusted to 1× SDS-SB (62.5 mM Tris-HCl, pH 6.8, 10% glycerol, 2% SDS, 0.02% (w/v) Bromophenol Blue, 100 mM DTT), heated to 95° C. for 5 min, and resolved by SDS-polyacrylamide gel electrophoresis. Gel filtration and Dicer activity assays were as described (Nykänen et al., 2001).

M. Photocleavage and Protein Recovery

90 pmoles of siRNA containing a 5′ PC-biotinylated linker (Glen Research) was conjugated to 600 ul M-280 Dyna-beads (Dynal) by incubation at 4° C. The paramagnetic beads were incubated for 60 minutes at 25° C. in a 1 ml standard RNAi reaction. After incubation, beads were captured on a Dynal MPC-E magnetic stage, washed 4 times in ice-cold lysis buffer containing 0.1% (w/v) NP-40, resuspended in 200 μl icecold lysis buffer, transferred to a 96-well plate (30 ul per well) immersed in ice and photocleaved by irradiation for 20 minutes with a 302 nm light source ˜7.5 cm from the samples. Beads were captured with the magnet and the supernatant dialyzed against 2% (v/v) glacial acetic acid in a 10 kDa cut-off dialysis cup (Pierce) for 12-16 hours. The dialysate was lyophilized, resuspended in 2×SDS-SB, resolved by electrophoresis through an 8% SDS-polyacrylamide gel, and stained with silver. Bands were excised, digested with trypsin in situ, and the resulting peptides analyzed by electrospray mass spectrometry.

N. Immunoprecipitation and Western Blotting

Normal rabbit, α-Dcr-2, and α-R2D2 antisera were first bound to Protein A agarose beads for 2 hours at 4° C. in lysis buffer. After washing with RIPA buffer (150 mM NaCl, 1% (v/v) NP40, 0.5% (w/v) sodium deoxycholate, 0.1% SDS, 25 mM Tris-HCl, pH 7.6), the beads (25 μl) were incubated with 15 μl of crosslinked lysate for 2 hours at 4° C. After washing in RIPA buffer, the beads were boiled in 1×SDS-SB and the eluted proteins resolved by SDS-PAGE. For Western Blotting, the complexes were excised from the native gel, boiled in 1× SDS-SB at 95° C. for 10 minutes, and resolved by SDS-PAGE. Western Blotting was performed with 1:1000 dilution for α-Dcr-2 antisera and 1:5000 for α-R2D2.

### REFERENCES

Ambros, V. (2003). Cell 113, 673-676.
Aravin, et al. (2001). Curr Biol 11, 1017-1027.
Aza-Blanc, P., et al. (2003). Mol Cell 12, 627-637.
Bashirullah, A., et al. (1998). Annu Rev Biochem 67, 335-394.
Bassell, G. J., et al. (1999). Faseb J 13, 447-454.
Belloni, M., et al. (2002). Genetics 161, 1551-1559.
Berleth, T., et al. (1988). Embo J 7, 1749-1756.
Bernstein, E., et al. (2001). RNA 7, 1509-1521.
Billy, E., et al. (2001). Proc Natl Acad Sci USA 98, 14428-14433.
Boutla, A., et al. (2001). Curr Biol 11, 1776-1780.
Brendza, R. P., et al. (2002). Curr Biol 12, 1541-1545.
Brendza, R. P., et al. (2000). Science 289, 2120-2122.
Brennecke, J., et al. (2003). Cell 113, 25-36.
Caplen, N. J., et al. (2001). Proc Natl Acad Sci USA 98, 9742-9747.
Castagnetti, S., and Ephrussi, A. (2003). Development 130, 835-843.
Catalanotto, C., et al. (2002). Genes Dev 16, 790-795.
Caudy, A. A., et al. (2003). Nature 425, 411-414.
Caudy, A. A., et al. (2002). Genes Dev 16, 2491-2496.
Celotto, A. M., and Graveley, B. R. (2002). RNA 8, 718-724.
Cha, B. J., et al. (2001). Cell 106, 35-46.
Cha, B. J., et al. (2002). Nat Cell Biol 4, 592-598.
Chiu, Y.-L., and Rana, T. M. (2002). Molecular Cell 10, 549-561.
Chou, T. B., and Perrimon, N. (1996). Genetics 144, 1673-1679.
Clegg, N. J., et al. (2001). Dev Genes Evol 211, 44-48.
Clegg, N. J., et al. (1997). Development 124, 4661-4671.
Cogoni, C., and Macino, G. (1997). Proc Natl Acad Sci USA 94, 10233-10238.
Consortium, T. F. (2003). Nucleic Acids Research 31, 172-175.
Cooperstock, R. L., and Lipshitz, H. D. (2001). Int Rev Cytol 203, 541-566.
Dalmay, T., et al. (2000). Cell 101, 543-553.
Dalmay, T., et al. (2001). Embo J 20, 2069-2078.
Deng, W., and Lin, H. (1997). Dev Biol 189, 79-94.
Doench, J. G., et al. (2003). Genes Dev 17, 438-442.
Doi, N., et al. (2003). Curr Biol. 13, 41-46.
Driever, W., and Nusslein-Volhard, C. (1988). Cell 54, 83-93.
Duncan, J. E., and Warrior, R. (2002). Curr Biol 12, 1982-1991.
Eddy, E. M. (1975). Int Rev Cytol 43, 229-280.
Elbashir, S. M., et al. (2001 a). Nature 411, 494-498.
Elbashir, S. M., et al. (2001b). Genes Dev 15, 188-200.
Elbashir, S. M., et al. (2001c). EMBO J 20, 6877-6888.
Ephrussi, A., et al. (1991). Cell 66, 37-50.
Ephrussi, A., and Lehmann, R. (1992). Nature 358, 387-392.
Fagard, M., et al. (2000). Proc Natl Acad Sci USA 97, 11650-11654.
Findley, S. D., et al. (2003). Development 130, 859-871.
Fire, A., et al. (1998). Nature 391, 806-811.
Gillespie, D. E., and Berg, C. A. (1995). Genes Dev 9, 2495-2508.
Gonzalez-Reyes, A., et al. (1995). Nature 375, 654-658.
Gonzalez-Reyes, A., et al. (1997). Development 124, 4927-4937.
Grishok, A., et al. (2001). Cell 106, 23-34.
Grishok, A., et al. (2000). Science 287, 2494-2497.
Gvozdev, V. A., et al. (2003). Genetica 117, 239-245.
Haley, B., et al. (2003). Methods 30, 330-336.
Hamilton, A. J., and Baulcombe, D. C. (1999). Science 286, 950-952.
Hamilton, B. A., and Zinn, K. (1994). Methods Cell Biol 44, 81-94.
Hammond, S. M., et al. (2000). Nature 404, 293-296.
Hammond, S. M., et al. (2001). Science 293, 1146-1150.
Harlow, E., and Lane, D. (1999). In Using Antibodies: A Laboratory Manual (Cold Spring Harbor Laboratory Press), pp. 70-80.
Himber, C., et al. (2003). EMBO J 22, 4523-4533.
Hutvágner, G., et al. (2004). PLoS Biology, in press.
Hutvágner, G., and Zamore, P. D. (2002). Science 297, 2056-2060.
Hutvágner, G., et al. (2001). Science 293, 834-838.
Ikenishi, K. (1998). Dev Growth Differ 40, 1-10.
Ishizuka, A., et al. (2002). Genes Dev 16, 2497-2508.
Januschke, J., et al. (2002). Curr Biol 12, 1971-1981.
Kennerdell, J. R., et al. (2002). Genes Dev 16, 1884-1889.
Kennerdell, J. R., and Carthew, R. W. (1998). Cell 95, 1017-1026.
Ketting, R. F., et al. (2001). Genes Dev 15, 2654-2659.
Ketting, R. F., et al. (1999). Cell 99, 133-141.
Khvorova, A., et al. (2003). Cell 115, 209-216.
Kim-Ha, J., et al. (1995). Cell 81, 403-412.
Kim-Ha, J., et al. (1993). Development 119, 169-178.
Kim-Ha, J., et al. (1991). Cell 66, 23-35.
Knight, S. W., and Bass, B. L. (2001). Science 293, 2269-2271.
Kolonin, M. G., and Finley, R. L. J. (2000). Dev Biol 227, 661-672.
Koonin, E. V. (1992). Trends Biochem Sci 17, 495-497.
Kooter, J. M., et al. (1999). Trends Plant Sci 4, 340-347.
Lagos-Quintana, M., et al. (2003). RNA 9, 175-179.
Lagos-Quintana, M., et al. (2002). Curr Biol 12, 735-739.
Lagos-Quintana, M., et al. (2001). Science 294, 853-858.
Lee, R. C., and Ambros, V. (2001). Science 294, 862-864.
Lee, R. C., et al. (1993). Cell 75, 843-854.
Lehmann, R., and Nusslein-Volhard, C. (1986). Cell 47, 141-152.
Liang, L., et al. (1994). Development 120, 1201-1211.
Lieberfarb, M. E., et al. (1996). Development 122, 579-588.
Lim, L. P., et al. (2003a). Science 299, 1540.
Lim, L. P., et al. (2003b). Genes Dev 17, 991-1008.
Lin, H., et al. (1994). Development 120, 947-956.
Linder, P., and Daugeron, M. C. (2000). Nat Struct Biol 7, 97-99.
Liu, Q., et al. (2003). Science 301, 1921-1925.
Lohmann, J. U., et al. (1999). Dev Biol 214, 211-214.
Markussen, F. H., et al. (1995). Development 121, 3723-3732.
Martinez, J., et al. (2002). Cell 110, 563-574.
Micklem, D. R., et al. (1997). Curr Biol 7, 468-478.
Mohr, E., and Richter, D. (2001). Int J Biochem Cell Biol 33, 669-679.
Mooslehner, K., et al. (1991). Mol Cell Biol 11, 886-893.
Morel, J. B., et al. (2002). Plant Cell 14, 629-639.
Mourelatos, Z., et al. (2002). Genes Dev 16, 720-728.
Mourrain, P., et al. (2000). Cell 101, 533-542.
Myers, J. W., et al. (2003) Nat Biotechnol. 21, 324-8.
Nakamura, A., et al. (2004). Dev Cell 6, 69-78.
Neuman-Silberberg, F. S., and Schupbach, T. (1996). Mech Dev 59, 105-113.
Neuman-Silberberg, F. S., and Schupbach, T. (1993). Cell 75, 165-174.
Ngo, H., et al. (1998). Proc Natl Acad Sci U S A 95, 14687-14692.
Nykänen, A., et al. (2001). Cell 107, 309-321.
O'Connor, M., and Chia, W. (1993). In Methods Mol Biol (Totowa, N.J., Humana Press Inc), pp. 75-85.
Olsen, P. H., and Ambros, V. (1999). Dev Biol 216, 671-680.
Paddison, P. J., et al. (2002). Genes Dev 16, 948-58.
Pal-Bhadra, M., et al. (2002). Mol Cell 9, 315-327.
Palacios, I. M., and St Johnston, D. (2002). Development 129, 5473-5485.
Palatnik, J. F., et al. (2003). Nature 425, 257-263.
Palauqui, J. C., et al. (1997). EMBO J 16, 4738-4745.
Park, W., et al. (2002). Curr Biol 12, 1484-1495.
Provost, P., et al. (2002). Proc Natl Acad Sci USA. 99, 16648-53.
Ratcliff, F. G., et al. (1999). Plant Cell 11, 1207-1216.
Reinhart, B. J., et al. (2002). Genes Dev 16, 1616-1626.
Riechmann, V., and Ephrussi, A. (2001). Curr Opin Genet Dev 11, 374-383.
Riechmann, V., et al. (2002). Nat Cell Biol 4, 337-342.
Roignant, J. Y., et al. (2003). RNA 9, 299-308.
Rongo, C., et al. (1995). Development 121, 2737-2746.
Rorth, P. (1998). Mech Dev 78, 113-118.
Roth, S., et al. (1995). Cell 81, 967-978.
Salles, F. J., et al. (1994). Science 266, 1996-1999.
Sánchez-Alvarado, A., and Newmark, P. A. (1999). Proc Natl Acad Sci USA 96, 5049-5054.
Saxena, S., et al. (2003). J Biol Chem. 278, 44312-9.
Schmidt, A., et al. (1999). Genetics 151, 749-760.
Schramke, V., and Allshire, R. (2003). Science 301, 1069-1074.
Schwarz, D. S., et al. (2003) Cell 115, 199-208.
Schwarz, D. S., et al. (2002). Molecular Cell 10, 537-548.
Schupbach, T., and Wieschaus, E. (1991). Genetics 129, 1119-1136.
Seggerson, K., et al. (2002). Dev Biol 243, 215-225.
Shulman, J. M., et al. (2000). Cell 101, 377-388.
Sijen, T., and Plasterk, R. H. (2003). Nature 426, 310-314.
St Johnston, D., et al. (1989). Development 107, 13-19.
Stapleton, W., et al. (2001). Chromosoma 110, 228-240.
Stark, A., et al. (2003). PLoS Biology 1, 397-409.
Struhl, G., et al. (1989). Cell 57, 1259-1273.
Tabara, H., et al. (2002). Cell 109, 861-871.
Tabara, H., et al. (1999). Cell 99, 123-132.
Tang, G., et al. (2003). Genes Dev 17, 49-63.
Tanner, N. K., and Linder, P. (2001). Mol Cell 8, 251-262.
Theodosiou, N. A., and Xu, T. (1998). Methods 14, 355-365.
Theurkauf, W. E. (1994). Methods Cell Biol 44, 489-505.
Theurkauf, W. E., et al. (1993). Development 118, 1169-1180.
Theurkauf, W. E., et al. (1992). Development 115, 923-936.
Tijsterman, M., et al. (2002a). Science 295, 694-697.
Tijsterman, M., et al. (2002b). Annu Rev Genet 36, 489-519.
Tracey, W. D. J., et al. (2000). Genetics 154, 273-284.
Tuschl, T., et al. (1999). Genes Dev 13, 3191-3197.
van Eeden, F., and St Johnston, D. (1999). Curr Opin Genet Dev 9, 396-404.
Vanzo, N. F., and Ephrussi, A. (2002). Development 129, 3705-3714.
Vaucheret, H., and Fagard, M. (2001). Trends Genet 17, 29-35.
Voinnet, 0. (2002). Curr Opin Plant Biol 5, 4444-4451.
Volpe, T. A., et al. (2002). Science 297, 1833-1837.
Waterhouse, P. M., et al. (1998). Proc Natl Acad Sci USA 95, 13959-13964.
Webster, P. J., et al. (1997). Genes Dev 11, 2510-2521.
Weng, Y., et al. (1996). Mol Cell Biol 16, 5477-5490.
Wianny, F., and Zernicka-Goetz, M. (2000). Nat Cell Biology 2, 70-75.
Wightman, B., et al. (1993). Cell 75, 855-862.
Wilhelm, J. E., et al. (2003). J Cell Biol 163, 1197-1204.
Williams, R. W., and Rubin, G. M. (2002). Proc Natl Acad Sci USA 99, 6889-6894.
Wilson, J. E., et al. (1996). Development 122, 1631-1639.
Winston, W. M., et al. (2002). Science 295, 2456-2459.
Wu-Scharf, D., et al. (2000). Science 290, 1159-1162.
Xie, Z., et al. (2003). Curr Biol 13, 784-9.
Zaccai, M., and Lipshitz, H. D. (1996). Dev Genet 19, 249-257.
Zamore, P. D., et al. (2000). Cell 101, 25-33.
Zeng, Y., et al. (2003). Proc Natl Acad Sci USA 100, 9779-9784.
Zeng, Y., et al. (2002). Mol Cell 9, 1327-1333.
Zhang, H., et al. (2002). EMBO J 21, 5875-85.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for identifying an RNAi modulatory compound, comprising,
contacting a cell expressing an Armi polypeptide or a fragment thereof with a test compound, and
determining the ability of the test compound to modulate an Armi activity,
such that the RNAi modulatory compound is identified.

2. A method for identifying an RNAi modulatory compound, comprising,
contacting a composition comprising an Armi polypeptide or a fragment thereof with a test compound, and
determining the ability of the test compound to modulate an Armi activity,
such that the RNAi modulatory compound is identified.

3. The method of claim 2, wherein the composition is a cell extract.

4. A method for identifying an RNAi modulatory compound, comprising,
contacting an organism expressing an Armi polypeptide or a fragment thereof with a test compound, and
determining the ability of the test compound to modulate an Armi activity,
such that the RNAi modulatory compound is identified.

5. The method of any of claims 1-4, wherein determining the ability of the test compound to modulate an Armi activity comprises at least one of the following: detecting Armi helicase activity, detecting RISC assembly, detecting RISC activation, and detecting siRNA or miRNA processing for incorporation into a RISC.

6. The method of any of claims 1-4, wherein determining the ability of the test compound to modulate an Armi activity comprises detecting the interaction of Armi peptide with a peptide selected from the group comprising the molecular components of RISC (e.g. Argonaute polypeptides such as Aubergine) and RISC-interacting polypeptides and fragments thereof.

7. The method of any of claims 1-4, wherein determining the ability of the test compound to modulate an Armi activity comprises detecting RISC assembly.

8. The method of any of claims 1-7, wherein detecting RISC assembly comprises detecting the interaction of Armi peptide with a peptide selected from the group consisting of the molecular components of RISC (e.g. Dcr-2 and Argonaute polypeptides such as Aubergine and Argonaute 2).

9. The method of any of claims 1-4, wherein determining the ability of the test compound to modulate an Armi activity comprises detecting the interaction of Armi peptide with single-stranded products of siRNA unwinding.

10. The method of any of claims 1-4, wherein determining the ability of the test compound to modulate an Armi activity comprises detecting accumulation of a RISC assembly intermediate complex (e.g. complex B or complex A).

11. The method of any of claims 1-4, wherein determining the ability of the test compound to modulate an Armi activity comprises detecting activation of RISC.

12. The method of claim 11, wherein detecting activation of RISC comprises detecting interaction of an Armi peptide or fragment thereof with single-stranded products of siRNA unwinding.

13. The method of any of claims 1-4, wherein determining the ability of the test compound to modulate an Armi activity comprises detecting Armi helicase activity.

14. The method of any of claims 1-13, wherein the compound is selected from the group consisting of a small molecule, a peptide, a polynucleotide, an antibody or biologically active portion thereof, a peptidomimetic, and a non-peptide oligomer.

15. The method of any of claims 5, 7, 8, 10 or 11, wherein detecting RISC assembly or RISC activation comprises detecting the cleavage state of a detectably labeled reporter RNA.

16. The method of claim 15, wherein the reporter RNA is radioactively labeled.

17. The method of claim 15, wherein the reporter RNA is fluorescently labeled.

18. The method of either of claims 1 or 3, wherein determining the ability of the test compound to modulate Armi activity comprises detecting at least one of the following: disrupted microtubule organization, disrupted localization of a polynucleotide or peptide, and premature expression of a polynucleotide.

19. The method of either of claims 1 or 3, wherein determining the ability of the test compound to modulate Armi activity comprises detecting the localization or abundance of a polynucleotide or peptide selected from the group consisting of oskar mRNA, Oskar peptide, gurken mRNA, Gurken peptide, vasa mRNA, Vasa peptide, spn-E mRNA, Spn-E protein, stellate mRNA and Stellate peptide.

20. The method of any one of claims 1-19, further comprising comparing to a suitable control the ability of the test compound to modulate Armi activity.

21. The method of claim 1, wherein the cell expressing an Armi polypeptide or a fragment thereof is selected from the group consisting of embryonic cells, ovarian cells, Drosophila melanogaster cells, Drosophila melanogaster cell lines, mammalian cells, and mammalian cell lines.

22. The method of claim 2, wherein the composition comprising an Armi polypeptide or a fragment thereof is selected from the group consisting of embryonic cell lysates, ovarian cell lysates, Drosophila melanogaster cell lysates, Drosophila melanogaster cell line lysates, mammalian cell lysates and mammalian cell line lysates.

23. The method of any of claims 1-22, wherein the Armi polypeptide is selected from the group consisting of SDE3 (Arabidopsis thaliana), Armi (D. melanogaster), and Mov10 (mammalian).

24. An assay for detecting modulation of RNA interference, comprising,
contacting a reaction mixture comprising ovary lysates with a test siRNA, and
evaluating the effect of the test siRNA on an indicator of RNA interference,
such that modulation of RNA interference is detected.

25. The assay of claim 24, wherein the indicator of RNA interference is a detectably labeled reporter RNA comprising a region complementary to a strand of the test siRNA.

26. The assay of claim 25, wherein the reporter RNA is radioactively labeled.

27. The assay of claim 25, wherein the reporter RNA is fluorescently labeled.

28. The assay of either of claims 26 or 27, wherein detection of cleavage of the detectably labeled reporter RNA indicates RNA interference.

29. The assay of any one of claims 24-28, wherein the reaction mixture comprises Drosophila melanogaster ovary lysates containing Armi polypeptide or a fragment thereof.

30. The assay of any one of claims 24-28, wherein the reaction mixture comprises armi mutant or Armi-depleted Drosophila melanogaster ovary lysates.

31. The assay of any one of claims 29 or 30, wherein the composition further comprises a Mov10 polypeptide or a fragment thereof.

32. A method for identifying developmental factors, comprising,
comparing armi mutant or Armi-depleted embryos, cells or cell extracts to wild-type or control embryos, cells or cell extracts,
such that developmental factors are identified.

33. The method of claim 32, wherein at least one potential developmental factor is compared between armi mutant or Armi-depleted embryos, cells or cell extracts and wild-type or control embryos, cells or cell extracts.

34. The method of claim 33, wherein the abundance of at least one potential developmental factor is examined.

35. The method of claim 33, wherein the localization or distribution of at least one potential developmental factor is examined.

36. The method of claim 34 or 35, wherein potential developmental factor mRNA, mRNA fragment, polypeptide, or polypeptide fragment is examined.

37. A method for identifying a compound suitable for modulating RNA interference in a cell, comprising,
contacting an armi mutant or Armi-depleted cell or cell extract with a test compound, and
determining the ability of the test compound to modulate an Armi activity,
such that the modulator of RNA interference is identified.

38. A method for identifying a compound suitable for modulating RNA interference in a cell, comprising,
contacting an armi mutant or knockout cell or cell extract expressing a mutant or wild-type form of Armi polypeptide or fragment thereof from a transgenic or autonomously-replicating vector with a test compound, and
determining the ability of the test compound to modulate an Armi activity,
such that the modulator of RNA interference is identified.

39. A method for identifying an Armi form suitable for modulating RNA interference in a cell, comprising,
expressing a mutant or wild-type form of Armi polypeptide or fragment thereof via a transgenic or autonomously-replicating vector within an organism, embryo, cell or cell extract, and
determining the ability of Armi polypeptide or fragment thereof to modulate RNA interference,
such that the modulator of RNA interference is identified.

40. The method of claim 39, wherein the Armi polypeptide or fragment is selected from the group consisting of SDE3 (Arabidopsis thaliana), Armi (D. melanogaster), and Mov10 (mammalian).

41. The method of claim 39, wherein the organism, embryo, cell or cell extract comprises an armi mutant or knockout organism, embryo, cell or cell extract.

42. A method of modulating RNA interference in a subject comprising administering to the subject an RNA interference modulator identified according to the methods of any one of claims 1-23 or claims 37-41, in an amount effective to modulate RNA interference.

43. A method of modulating RISC activity or assembly in a subject comprising administering to the subject an RNA interference modulator identified according to the methods of any one of claims 1-23 or claims 37-41, in an amount effective to modulate RISC activity or assembly.

44. A pharmaceutical composition comprising an RNA interference modulator identified by any one of claims 1-23 or claims 37-43.

45. A method of treating an RNA interference disease or disorder comprising administering the pharmaceutical composition of claim 44.

46. A method of treating cancer comprising administering the pharmaceutical composition of claim 44.


SEQUENCE LISTING (25 sequences)

SEQ ID NO: 1 - protein, 1274 aa, Drosophila melanogaster
MSSEASGSITIKEEASTSGTATNIPVESSETEAKKPEIGADKAESQSETTEGGKAAETTENHLKRLQNLRKELSYLSETDWMYESLDKKAAHKFFTNPDRNREDILESLDRENSFLDQKLMEEKMDQQLKANPNEINGVLSNKIAELTHGLSEMDVSKESSCTARKGVITSLDGDRGVIDKDVLFETKVAEDIILDLHVGCVVEYLTFTTGEAMRVVKVKSILEHSWEDTSQKEIEKAVDNLKNEKPTFFNTETRSVLGLISQRLASSIDVETEYGQLTVELDNIEMNFIPTNGDRVRLECNIQLDDGFVDKQGEILEVTKLFPTRIQEGEKCIVERVYVHMVVLGPETYILKTDLPTGTDLHLGDIVLADLIECQYSKFTRRAIKITPLEKNFGATKLTQQSSMGSSGSGKAVTVTGVNRFITAELWQKESVSLKLTNNLNRTLRLESITVCNDSESQLSVVSPLESKEISSGSEITVTFEIHTQFLGEAIEKYVLNFDLLKVRRVFTVIVCKTKEEVAEAEKRMIAAEALMAPGRNSQERSRFYANQVWCNKVDVIPGQQIVTKRRFVALRLGCFEVPKELRQICLTSERRQEMIKAIEQHYSFLTEPLSIKTYMHRFRLLLHLEEIECFVNFRNYDRDRAHFLRDGEFLTLQIENLAERRPSLVIGDTLRVINPWSDPDSQTTKSYEGIIHKVLFDRILLKFHSSFQEKYNGEDYRLEFYFSRYSFRKQHHAISKIVGVMGEDFLFPSKVTKRENPQLDVYMKDDDMYLYDSKLEWYNQSLNSIQKRAVFNILRGEAENIPYVLFGPPGSGKTMTLIETLLQLVRNLPGARILVGTPSNSSADLVTKRLIDSKALLQGDFIRLVSYNQVEKDLIPPEIMSYCATSDVGAVGSCEDKMMVTESGLKLRCQAKFIGTHRITISTCTTLGNFLQLGFPAGHFTHVLFDEAGQCTEPETMVPIVMLTKKRSQVVLSGDPRQLQSIVTSRIASKMGFSISFLERLLERSPYRKDLQRFPESSGYNPLVLTKLLYNYRALPSIMSIYSRLFYDDELIPVLSEKDSRESRLLSKLRCVFESEKDIPQAHGTFFYGIIGENRQNNDSPSWFNPQEVREVFLMTIALYRANVTADQIGIITPYQKQVKMLRSMFIGTDVVMPKIGSVEEFQGQERDIILISTVRSSEEILRMDARFSLGFVRCSKRLNVAVSRARAMMIIFGNPHLLAVDECWRQLILFCVKNNAYFGCDLPQMVINQKDEVPVCLETFVPSLNTTDDLN

SEQ ID NO: 2 - protein, 1002 aa, Arabidopsis thaliana
MSVSGYKSDDEYSVIADKGEIGFIDYQNDGSSGCYNPFDEGPVVVSVPFPFKKEKPQSVTVGETSFDSFTVKNTMDEPVDLWTKIYASNPEDSFTLSILKPPSKDSDLKERQCFYETFTLEDRMLEPGDTLTIWVSCKPKDIGLHTTVVTVDWGSDRVERVVFLLAEDKISSSLTSNRPYSRSRRAPKKDFAVDDYVKGSRPSKVVERSFRNRLPLYEIPKEIREMIENKEFPDDLNEGLTARNYANYYKTLLIMEELQLEEDMRAYDMENVSMKRRGIYLSLEVPGLAERRPSLVHGDFIFVRHAYDDGTDHAYQGFVHRVEADEVHMKFASEFHQRHTAGSVYNVRFTYNRINTRRLYQAVDAAEMLDPNFLFPSLHSGKRMIKTKPFVPISPALNAEQICSIEMVLGCKGAPPYVIHGPPGTGKTMTLVEAIVQLYTTQRNARVLVCAPSNSAADHILEKLLCLEGVRIKDNEIFRLNAATRSYEEIKPEIIRFCFFDELIFKCPPLKALTRYKLVVSTYMSASLLNAEGVNRGHFTHILLDEAGQASEPENMIAVSNLCLTETVVVLAGDPRQLGPVIYSRDAESLGLGKSYLERLFECDYYCEGDENYVTKLVKNYRCHPEILDLPSKLFYDGELVASKEDTDSVLASLNFLPNKEFPMVFYGIQGCDEREGNNPSWFNRIEISKVIETIKRLTANDCVQEEDIGVITPYRQQVMKIKEVLDRLDMTEVKVGSVEQFQGQEKQVIIISTVRSTIKHNEFDRAYCLGFLSNPRRFNVAITRAISLLVIIGNPHIICKDMNWNKLLWRCVDNNAYQGCGLPEQEEFVEEPFKQEGSSNGPQYPPEAEWNNSGELNNGGANENGEWSDGWNNNGGTKEKNEWSDGWNSNGGGTKKKDEWSDGWDNNGGTNGINQEGSSNAPQDPQEAEWNDSGEVKNGGTKEKDVRSDGWNNNGGKNEKEECCDGWKDGGSGEEIKNGGKFETRGDFVAKEEDEWSDGWK

SEQ ID NO: 3 - protein, 1004 aa, Mus musculus
MPSKFSCRKLRETGQRFESFLAERGLDLETDRERLRTIYNHDFKPSYGTPAPGFSSMLYGMKIANLAFVTKTRVRFFKLDRWADVQLPEKRRIKPGSNISKQHRSLLARIFHDRAEYLHGKHGVDVEVQGPHEARDGQLLIRLDLNRKEVLTLRLRNGGSKPVTLTHLFPLCWTPQFVFYHGEQDLPCPLGPGESYELHIYCKTSIVGYFPATVLWELLGPGESGAEGAETFYIARFLAAVAHSPLAAQLKPTTPFKRPPRLTRNSVLTNRIEEGERPDRAKGYELELSLALGTYYPPILLRQLLPTLLQGPSIFTAPKEVAEIKAQLETTLKSRNYEVKLRLLLHLEELQMEHDIRHYDLDSVPMTWDPVDQNPRLLTLEVPGVAESRPSVLRGDHLFALLSSETQQDDPVTYKGFVHKVELDRVKLSFSTSLLSRFVDGLTFKVNFTFNRQPLRVQHRALELTGRWVLWPMLFPVASRGVSLLPSDVKFKLYDRSLESNPEQLQAMKHIVRGTTRPAPYIIFGPPGTGKTVTLVEAIKQVVKHLPKAHILACAPSNSGADLLCQRLRVHLPSSIYRLLAPSRDIRMVPEDIKTCCNWDAKKGEYVYPAKKHLQQYRVLITTLITASRLVSAQFPIDHFTHIFIDEAGHCMEPESLVAIAGLMDVKETGNPGGQLVLAGDPRQLGPVLRSPLALKHGLGYSLLERLLAYNSLYKKGPNGYDPQFITKLLRNYRSHPTILDIPNQLYYDGELQACADVVDRERFCRWEGLPQQGFPIIFHGVMGKDEREGNSPSFFNPEEAATVTSYLKQLLAPSSKKGKARLSPRNVGVISPYRKQVEKIRYCITKLDRELRGLDDIKDLKVGSVEEFQGQERSVILISTVRSSQSFVQLDLDFNLGFLKNPKRFNVAVTRAKALLIVVGNPLLLGHDPDWKTFLEFCKENGGYTGCPFPAKLDLQQGQDLLQGLSKLSPSTSGPRRHQNLPQEREGEGGLPLQVEPEWRNEL

SEQ ID NO: 4 - protein, 1003 aa, Homo sapiens
MPSKFSCRQLREAGQCFESFLVVRGLDMETDRERLRTIYNRDFKISFGTPAPGFSSMLYGMKIANLAYVTKTRVRFFRLDRWADVRFPEKRRMKLGSDISKHHKSLLAKIFYDRAEYLHGKHGVDVEVQGPHEARDGQLLIRLDLNRKEVLTLRLRNGGTQSVTLTHLFPLCRTPQFAFYNEDQELPCPLGPGECYELHVHCKTSFVGYFPATVLWELLGPGESGSEGAGTFYIARFLAAVAHSPLAAQLKPMTPFKRTRITGNPVVTNRIEEGERPDRAKGYDLELSMALGTYYPPPRLRQLLPMLLQGTSIFTAPKEIAEIKAQLETALKWRNYEVKLRLLLHLEELQMEHDIRHYDLESVPMTWDPVDQNPRLLTLEVPGVTESRPSVLRGDHLFALLSSETHQEDPITYKGFVHKVELDRVKLSFSMSLLSRFVDGLTFKVNFTFNRQPLRVQHRALELTGRWLLWPMLFPVAPRDVPLLPSDVKLKLYDRSLESNPEQLQAMRHIVTGTTRPAPYIIFGPPGTGKTVTLVEAIKQVVKHLPKAHILACAPSNSGADLLCQRLRVHLPSSIYRLLAPSRDIRMVPEDIKPCCNWDAKKGEYVFPAKKKLQEYRVLITTLITAGRLVSAQFPIDHFTHIFIDEAGHCMEPESLVAIAGLMEVKETGDPGGQLVLAGDPRQLGPVLRSPLTQKHGLGYSLLERLLTYNSLYKKGPDGYDPQFITKLLRNYRSHPTILDIPNQLYYEGELQACADVVDRERFCRWAGLPRQGFPIIFHGVMGKDEREGNSPSFFNPEEAATVTSYLKLLLAPSSKKGKARLSPRSVGVISPYRKQVEKIRYCITKLDRELRGLDDIKDLKVGSVEEFQGQERSVILISTVRSSQSFVQLDLDFNLGFLKNPKRFNVAVTRAKALLIIVGNPLLLGHDPDWKVFLEFCKENGGYTGCPFPAKLDLQQGQNLLQGLSKLSPSTSGPHSHDYLPQEREGEGGLSLQVEPEWRNEL

SEQ ID NO: 5 - RNA, 2579 nt, Drosophila melanogaster
acuuuccuccaagcgauggccgcagucacaagugaauuccccagcaaaccgaucaguuauaccagcaccaauacuuccgccaaaaccuauuaucuuaaguccgugaaaaagcgggugaccacguguuuccagcaguugcgcgauaaacuccagucauccgguuccuuucgcaagaguuccuccagcugccucaaccagaucuucguaagguccgauuucuccgcaugcggcgaacgcuuucggaagaucuucaaaucggcgcgaaaaaccgaauugccggaguuguggaaggugccauugguggcccaugaacucaccagccggcagagcagccagcaguugcaagucguagccagacucuucucguccacucagauuuccaccaaggaaaucaccuacaacagcaacagcaacaccagcgagaacaacaugaccaucaucgagagcaacuauauauccgugcgcgaggaauaucccgauauagauagugaggugcgcgccauauugcugagccacgcccagaauggaaucacgauaucgagcaucaagagugaauaucgaaaacugacgggcaauccauuuccacugcacgacaacgugacggauuuccugcugaccauucccaaugugaccgcugagugcagcgaguccgguaagcggaucuucaaccugaaagcgagccugaagaacggucaccuccuggauauggugcucaaccagaaagagcgcaccagcgacuacagcagcggagcuccgucccuggagaacauaccacgagcaccuccacgcuacuggaagaaucccuucaaacggagggcucugucccagcugaacaccagcccgaggaccgugcccaagauaacggaugaaaagaccaaggauaucgccaccaggccgguuucgcugcaucaaauggccaaugaggcagcggagucgaacuggugcuaccaggauaauuggaagcaucucaacaauuucuaccagcaagccagcguaaaugcgccaaaaaugccaguacccaucaacaucuacagccccgaugccccagaggaaccaaucaauuuggcuccaccugggcaucagccaagcugcagaacccaaagccagaaaaccgaaccgacugaaaaccgccauuugggcaucuuugugcauccauuuaacggcaugaacauaaugaagagacgccacgaaaugacgcccacgccaacgauuuuaaccaguggaaccuacaacgauucucugcugacgauuaacucggauuacgaugccuaucugcuggacuuuccgcuuaugggcgaugauuuuaugcuauaucucgcccgaauggagcuaaaaugccgauuuaggcgucacgaacgcguccugcagucaggacuuuguguauccggacugacgaucaauggcgcccgaaaucguuuaaaaagaguccaauuacccgaggguacucagaucaucgucaauaucggaucgguggacauuaugcgcggcaagccuuugguucagaucgagcacgauuuucggcuacugaucaaggagaugcacaauaugcgauuggugccgauucuaacaaaucuugcaccgcugggcaacuauugucacgauaagguauuaugugacaaaaucuaccgauucaacaaguuuauccgaagcgaaugcugucaccuaaaggucaucgacauacacuccugucuaaucaacgaaaggggcguggugcgauuugauugcuuucaggccucaccacgccaagucaccgguuccaaggaacccuaucuguucuggaacaaaaucggucggcagcgcguacugcaaguuauugaaacgagucuggaguauuaaguuggguucuuaaucaagauacauauaugcaaauuuugacugggcuggcaccggaaccgacaaaauaagaacuuuuugaugauuuuacgauuuacgcugauggaucgcugcuuuuacggaauucgcuuaguuuuaauauguuuuauauguaguauguucucugucuuuguuuauuuauauguucgugcacuuguccuaguccauuauuguauauuauuguguguuuuguguucuauguuagauuuaaacuucucaauuuuucgcugucugugauuuguuuugccaaugccauugauuuucugcacacuuuuugcugcuaucccaaaagcuguguaaaauaaucaaaugcaaaauaagcgcaagcagcugaaaacuucucuucaaacuuuuccgcuuuucccaaaaccauuuugcuuugaaaucuguuuuuaccaaaucagauuaaacugcaaaauggaacuuaaaugcaaaucauugcaaugcuuauaaacuguuuuuuguucuauauacuuuugugugggucaaaauucggcaugcuccuguaucacacaaccugccacuugcccuuaaaaagaagggcgcagugggcgugguacguauacauaugagccaugcugcauuuuggccguaaugaaaaugcacugcuuuacuuggaaaauucgcuugcacaaaaucaacgccgcggcugauuuauuauugaugugcucaagcaaauucaagugaagcauuugcgcgauuuucgu

SEQ ID NO: 6 - RNA, 27 nt, Drosophila melanogaster
uguauuuacguugcauaugaaaugaua

SEQ ID NO: 7 - RNA, 29 nt, Drosophila pseudoobscura
cuacauuuuggcccuagacugaaaaugca

SEQ ID NO: 8 - RNA, 21 nt, Artificial Sequence
  synthetic construct
cguacgcggaauacuucgaaa

SEQ ID NO: 9 - RNA, 22 nt, Artificial Sequence
  synthetic construct
ucgaaguauuccgcguacguga

SEQ ID NO: 10 - RNA, 21 nt, Artificial Sequence
  synthetic construct
uguacgcggaauacuucgaaa

SEQ ID NO: 11 - DNA, 22 bp, Drosophila melanogaster
  RNA molecule with deoxythymidine at position 1
tcgaaguauuccgcguacguga

SEQ ID NO: 12 - protein, 1722 aa, Drosophila melanogaster
MEDVEIKPRGYQLRLVDHLTKSNGIVYLPTGSGKTFVAILVLKRFSQDFDKPIESGGKRALFMCNTVELARQQAMAVRRCTNFKVGFYVGEQGVDDWTRGMWSDEIKKNQVLVGTAQVFLDMVTQTYVALSSLSVVIIDECHHGTGHHPFREFMRLFTIANQTKLPRVVGLTGVLIKGNEITNVATKLKELEITYRGNIITVSDTKEMENVMLYATKPTEVMVSFPHQEQVLTVTRLISAEIEKFYVSLDLMNIGVQPIRRSKSLQCLRDPSKKSFVKQLFNDFLYQMKEYGIYAASIAIISLIVEFDIKRRQAETLSVKLMHRTALTLCEKIRHLLVQKLQDMTYDDDDDNVNTEEVIMNFSTPKVQRFLMSLKVSFADKDPKDICCLVFVERRYTCKCIYGLLLNYIQSTPELRNVLTPQFMVGRNNISPDFESVLERKWQKSAIQQFRDGNANLMICSSVLEEGIDVQACNHVFILDPVKTFNMYVQSKGRARTTEAKFVLFTADKEREKTIQQIYQYRKAHNDIAEYLKDRVLEKTEPELYEIKGHFQDDIDPFTNENGAVLLPNNALAILHRYCQTIPTDAFGFVIPWFHVLQEDERDRIFGVSAKGKHVISINMPVNCMLRDTIYSDPMDNVKTAKISAAFKACKVLYSLGELNERFVPKTLKERVASIADVHFEHWNKYGDSVTATVNKADKSKDRTYKTECPLEFYDALPRVGEICYAYEIFLEPQFESCEYTEHMYLNLQTPRNYAILLRNKLPRLAEMPLFSNQGKLHVRVANAPLEVIIQNSEQLELLHQFHGMVFRDILKIWHPFFVLDRRSKENSYLVVPLILGAGEQKCFDWELMTNFRRLPQSHGSNVQQREQQPAPRPEDFEGKIVTQWYANYDKPMLVTKVHRELTPLSYMEKNQQDKTYYEFTMSKYGNRIGDVVHKDKFMIEVRDLTEQLTFYVHNRGKFNAKSKAKMKVILIPELCFNFNFPGDLWLKLIFLPSILNRMYFLLHAEALRKRFNTYLNLHLLPFNGTDYMPRPLEIDYSLKRNVDPLGNVIPTEDIEEPKSLLEPMPTKSIEASVANLEITEFENPWQKYMEPVDLSRNLLSTYPVELDYYYHFSVGNVCEMNEMDFEDKEYWAKNQFHMPTGNIYGNRTPAKTNANVPALMPSKPTVRGKVKPLLILQKTVSKEHITPAEQGEFLAAITASSAADVFDMERLEILGDSFLKLSATLYLASKYSDWNEGTLTEVKSKLVSNRNLLFCLIDADIPKTLNTIQFTPRYTWLPPGISLPHNVLALWRENPEFAKIIGPHNLRDLALGDEESLVKGNCSDINYNRFVEGCRANGQSFYAGADFSSEVNFCVGLVTIPNKVIADTLEALLGVIVKNYGLQHAFKMLEYFKICRADIDKPLTQLLNLELGGKKMRANVNTTEIDGFLINHYYLEKNLGYTFKDRRYLLQALTHPSYPTNRITGSYQELEFIGDAILDFLISAYIFENNTKMNPGALTDLRSALVNNTTLACICVRHRLHFFILAENAKLSEIISKFVNFQESQGHRVTNYVRILLEEADVQPTPLDLDDELDMTELPHANKCISQEAEKGVPPKGEFNMSTNVDVPKALGDVLEALIAAVYLDCRDLQRTWEVIFNLFEPELQEFTRKVPINHIRQLVEHKHAKPVFSSPIVEGETVMVSCQFTCMEKTIKVYGFGSNKDQAKLSAAKHALQQLSKCDA

SEQ ID NO: 13 - DNA, 24 bp, Artificial Sequence
  synthetic construct
ccgtttcagattgctcctacacct

SEQ ID NO: 14 - DNA, 24 bp, Artificial Sequence
  synthetic construct
gcttaaggcgaatccaaacgaaat

SEQ ID NO: 15 - DNA, 24 bp, Artificial Sequence
  synthetic construct
tacgaaacgaggcgaatgaaatct

SEQ ID NO: 16 - DNA, 25 bp, Artificial Sequence
  synthetic construct
ggaatctgcctggtgcccgaatctt

SEQ ID NO: 17 - DNA, 25 bp, Artificial Sequence
  synthetic construct
gctccctcatggtcaaaaatatatc

SEQ ID NO: 18 - protein, 16 aa, Drosophila melanogaster
CLETFVPSLNTTDDLN

SEQ ID NO: 19 - RNA, 21 nt, Drosophila melanogaster
ugagguaguagguuguauagu

SEQ ID NO: 20 - RNA, 21 nt, Artificial Sequence
  synthetic construct
ucgaaguauuccgcguacgug

SEQ ID NO: 21 - RNA, 20 nt, Artificial Sequence
  synthetic construct
cgaaguauuccgcguacgug

SEQ ID NO: 22 - RNA, 20 nt, Artificial Sequence
  synthetic construct
cgaaguauuccgcguacgug

SEQ ID NO: 23 - RNA, 21 nt, Artificial Sequence
  synthetic construct
uauacaaccuacuaccucauu

SEQ ID NO: 24 - RNA, 31 nt, Artificial Sequence
  synthetic construct
caucacguacgcggaauacuucgaaaugucc

SEQ ID NO: 25 - protein, 13 aa, Arabidopsis thaliana
DLTEQLTFVVHNR